(19) United States
(12) Patent Application Publication
(10) Pub. No.: US 2025/0381276 A1
(43) Pub. Date: Dec. 18, 2025

(54) SMALL MOLECULE CONJUGATED CHARGE-ALTERING RELEASABLE TRANSPORTERS FOR NUCLEIC ACID DELIVERY

(71) Applicant: The Board of Trustees of the Leland Stanford Junior University, Stanford, CA (US)

(72) Inventors: Timothy Blake, Stanford, CA (US); Ronald Levy, Stanford, CA (US); Robert Waymouth, Stanford, CA (US); Paul Wender, Stanford, CA (US); Stefano Testa, Stanford, CA (US); Trevor Del Castillo, Stanford, CA (US); Ole Haabeth, Stanford, CA (US)

(73) Assignee: The Board of Trustees of the Leland Stanford Junior University, Stanford, CA (US)

(21) Appl. No.: 18/867,482

(22) PCT Filed: May 25, 2023
(86) PCT No.: PCT/US2023/023471

§ 371 Date: Nov. 20, 2024

Related U.S. Application Data
(60) Provisional application No. 63/346,014, filed on May 26, 2022.

(51) Int. Cl. A61K 47/59; A61K 9/00; A61K 47/54; A61P 37/06; C12N 15/88

(52) U.S. Cl. CPC A61K 47/593; A61K 9/0019; A61K 47/549; A61P 37/06; C12N 15/88

(57) ABSTRACT

There is provided herein a copolymer comprising a ligand moiety that binds to a cell surface receptor, one or two lipophilic polymer blocks and a poly(alpha aminoester) block for the delivery of therapeutic, diagnostic and imaging agents, including small molecules therapeutic agents and nucleic acids, into a cell.

### CROSS-REFERENCE TO RELATED APPLICATIONS

This application claims the benefit of U.S. Provisional Application No. 63/346,014, filed May 26, 2022, which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with Government support under contracts 1607092 awarded by the National Science Foundation and under contracts CA031845 and CA197353 awarded by the National Institutes of Health. The Government has certain rights in the invention.

### BACKGROUND

There is a need for new materials and strategies that enable or enhance the delivery of therapeutic agents, diagnostic probes and/or research tools across the plasma membrane of cells and other biological barriers, as required for a wide range of clinical, diagnostic and/or research applications. The delivery of such cargo, e.g., nuclei acids, has considerable clinical potential in connection with vaccination strategies for infectious diseases, cancer immunotherapy, protein therapy and gene editing. Provided herein are solutions to these and other problems in the art.

### BRIEF SUMMARY

In a first aspect, there is provided a copolymer, a cell-penetrating complex including a nucleic acid non-covalently bound to a copolymer, and a method of transfecting a nucleic acid into a cell.

In another aspect, provided is a co-polymer having a structure of Formula (I): comprising a ligand moiety, A, one or two lipophilic polymer blocks, LP¹, LP², and a poly (alpha aminoester) block, IM:

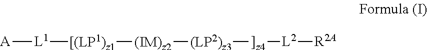

wherein:
   A is a ligand moiety that binds to a cell surface receptor;
   R^{2A} is hydrogen, halogen, —CCl₃, —CBr₃, —CF₃, —Cl₃, CHCl₂, —CHBr₂, —CHF₂, —CHl₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHC₂, —O CHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   L¹ and L² are optional and each L¹ and L² is independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
   LP¹ and LP² are each independently

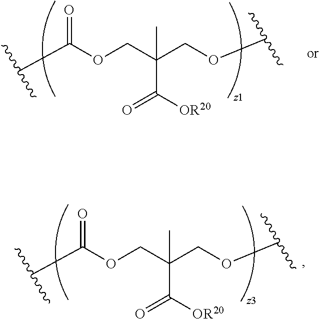

wherein
   z1 and z3 are independently from 1 to 100,
   wherein at least one of z1 or z3 is not 0; and
   R²⁰ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
IM is

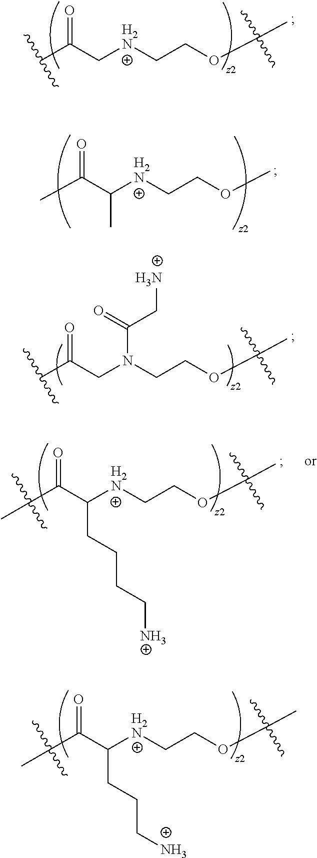

wherein z2 is from 2-100; and
z4 is 1 to 100.

In embodiments, A is saccharide, a disaccharide, an oligosaccharide, a liposaccharide, a lipid, a peptide, an antibody, or a small molecule. In embodiments, A is:

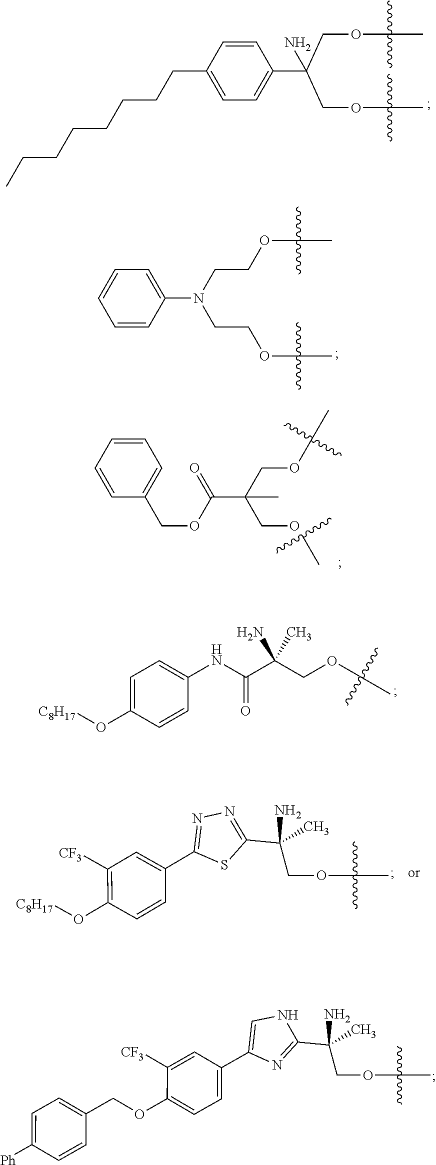

In embodiments, A is glucose (beta-D-glucopyranoside) or galactose (alpha-D-galactopyranose.

In embodiments, L₁ is:

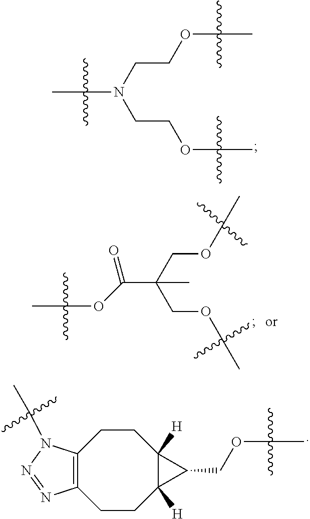

In embodiments, z1 is zero; z4 is 1; IM is

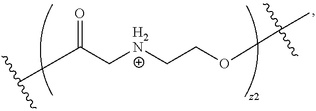

where z2 is from 1 to 100; and
LP₂ has the formula:

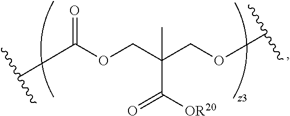

where z3 is from 1 to 100; and
   R²⁰ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, z2 is 12.

In embodiments, R²⁰ is independently hydrogen, unbranched C₁-C₃₀ alkyl, which may be fully saturated, mono- or polyunsaturated, or cholesterol.

In embodiments, R²⁰ is independently hydrogen, stearyl, oleyl, linoleyl, dodecyl, nonenyl, or cholesterol.

In embodiments, the copolymer has the structure:

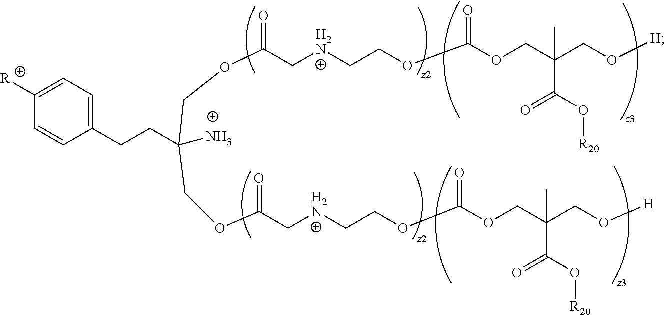

wherein
R³⁰ is unsubstituted C8 alkyl and
R²⁰ is independently hydrogen, unbranched C₁-C₃₀ alkyl, which may be fully saturated, mono- or polyunsaturated, or cholesterol, optionally wherein R²⁰ is independently hydrogen, stearyl, oleyl, linoleyl, dodecyl, nonenyl, or cholesterol.

In embodiments, the copolymer has a structure of Formula (Ia):

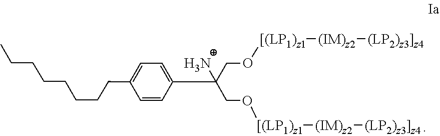

In embodiments, the copolymer has the structure of

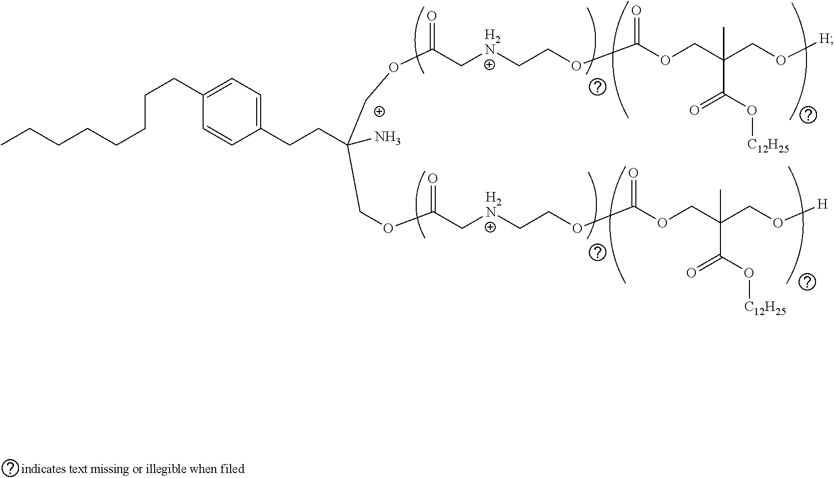

In embodiments, the copolymer has the structure of

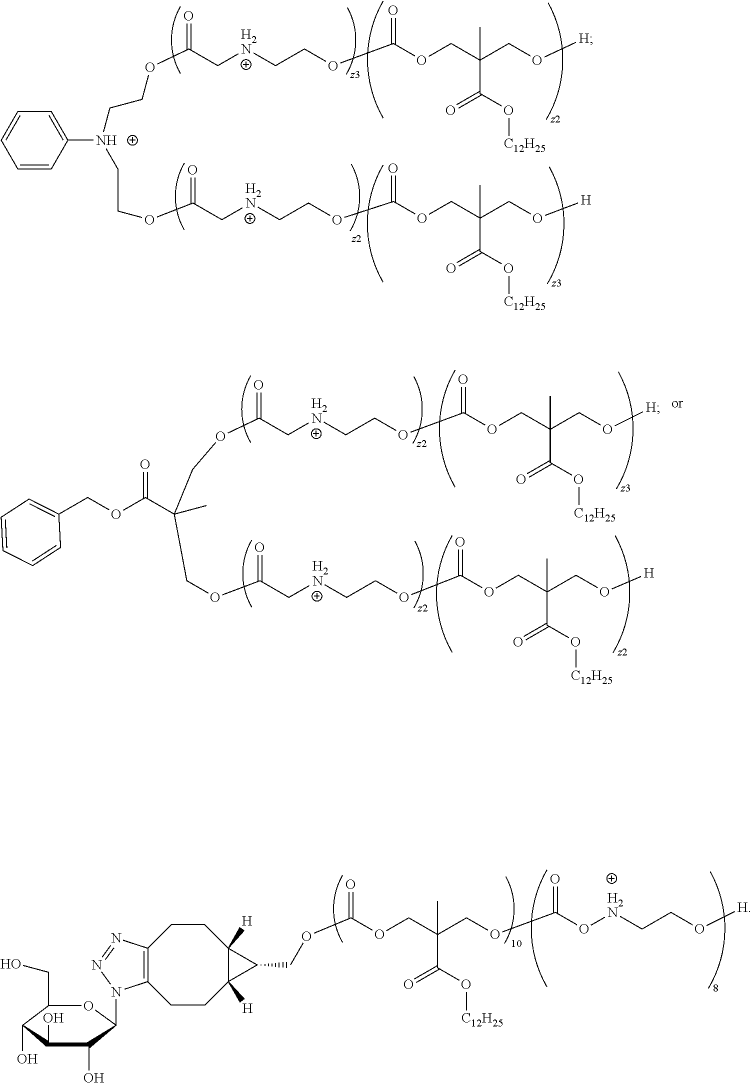

The present invention also provides a cell penetrating complex comprising a macromolecule, preferably a nucleic acid, non-covalently attached to a copolymer as described herein, optionally wherein the nucleic acid is an mRNA.

The present invention also provides a pharmaceutical composition comprising a cell-penetrating complex as described herein.

The present invention also provides a method of transfecting a nucleic acid into a cell, the method comprising contacting a cell with a cell penetrating complex as described herein, wherein the cell expresses a receptor on its surface that binds to the ligand moiety of the cell-penetrating complex, and wherein the contacting is performed ex vivo or in vivo.

In embodiments, the method comprises contacting a cell with a cell penetrating complex comprising a nucleic acid non-covalently attached to a copolymer described herein.

In embodiments, the cell penetrating complex comprises an mRNA non-covalently attached to the copolymer.

In embodiments, the cell penetrating complex comprises an mRNA non-covalently attached to a copolymer described herein, preferably the copolymer has a structure of Formula (Ia). In embodiments, the cell is a B or T lymphocyte. In embodiments, the cell is a marginal zone B cell, a follicular B cell, or a natural killer cell.

The present invention also provides a method for treating an autoimmune disease or disorder, a cancer, or an infectious disease, the method comprising administering a pharmaceutical composition comprising a cell-penetrating complex as described herein to a subject in need of therapy for an autoimmune disease or disorder, a cancer, or an infectious disease, optionally wherein administration is by a parenteral route, further optionally wherein administration is by an intravenous route.

In an aspect, provided is a cell penetrating complex comprising a macromolecule, such as a nucleic acid, non-covalently attached to the copolymer described herein.

In an aspect, provided is a method of transfecting a nucleic acid into a cell, the method comprising contacting a cell with the cell penetrating complex described herein.

Other aspect of the invention are disclosed infra.

### BRIEF DESCRIPTION OF DRAWINGS

FIG. 1A-B: A. Synthesis of CART-2: organocatalysts TU=1-(3,5bis(trifluoromethyl)phenyl)-3-cyclohexylthiourea, DBU=1,8-Diazabicyclo[5.4.0]undec-7-ene, (CART-1=Benzyl, D13: A11, PDI=1.25 Mn=6744 g/mol; CART-2=FTY720, A12: D12, PDI=1.20, Mn=6945 g/mol). DP determined by ¹H NMR, PDI determined by gel permeation chromatography. B. Synthesis of CART-3 and CART-4: Aryl/Lipophilic FTY720 analogue used for synthesis of CART-3: benzyl-2,2 bis(hydroxymethyl) propionate (B-DMPA); Aryl/Cationic FTY720 analogue used for synthesis of CART-4: N-phenyldiethanolamine (PhDEA). (CART-3=B-DMPA, A11:D11), PDI=1.27 Mn=6204 g/mol; CART-4=PhDEA, A14:D14), PDI=1.26, Mn=7792 g/mol). DP determined by 1H NMR, PDI determined by gel permeation chromatography.

FIG. 2A-F: CART-2 outperforms CART-1 in S1PR1-expressing cells but not in S1PR1-negative cells and pre-exposure to FTY720 impairs CART-2 transfection efficiency. Jurkat (A), K5620 (B) and CHO (C) cells were treated in vitro with Fluc mRNA complexed to either CART-2 or CART-1, with and without pre-treatment with free FTY720 for 30 minutes at 5 nM or 5 pM. Luminescence was measured 4 hours post-transfection. Data shows average relative luminescence units (RLUs) for 6 replicates (Jurkat and CHO) or 12 replicates (K562). Error bars show ±SD. K562 (D), CHO (E) and Jurkat cells (F) were treated in vitro with Fluc mRNA complexed to either the CART-3 or CART-4, with and without 30 minutes pre-treatment with free FTY720 at 5 nM. Luminescence was measured at 4 hours post-transfection. Data shows average RLUs for 6 replicates (CHO and K562) or 12 replicates (Jurkat). Error bars show ±SD. Statistical significance was calculated using an unpaired Student's t-test (*P<0.001; **P<0.05; ***P<0.01; ns, non-statistically significant).

FIG. 3A-D: CART-2 transfects primary human and murine lymphocytes more efficiently than CART-1. Data show the average % of EGFP+ cells in activated B and T murine splenic lymphocytes (A) and activated human B cells, CD4+ T cells and CD8+ T cells from healthy donor PBMCs (B) treated with EGFP mRNA complexed to either CART-1 (green) or CART-2 (blue). EGFP expression was calculated through flow cytometry with 2 replicates per group. Error bars show ±SD. Statistical significance was calculated using an unpaired Student's t-test (*P<0.001; ** P<0.05; *** P<0.01; ns, non-statistically significant). (C) Data shows the average Median Fluorescence Intensity (MFI) of a S1PR1-PE antibody on activated murine splenic T-cells (CD3+), B-cells (B220+), and total lymphocytes treated with either CART-1 or CART-2. The MFI of cells treated with a PE-isotype antibody was subtracted from each group to control for background fluorescence. Error bars show ±SD (n=2). Statistical significance was calculated using an unpaired Student's t-test (*P<0.001; ** P<0.05; *** P<0.01; ns, non-statistically significant). (D). Data shows average MFI of an S1PR1-APC antibody on activated human B-cells, CD4+ T-cells, CD8+ T-cells and total T-cells isolated from healthy donor PBMCs, treated with either CART-1 or CART-2. The MFI of cells treated with an APC-isotype antibody was subtracted from each group to control for background fluorescence. Error bars show ±SD (n=2). Statistical significance was calculated using an unpaired Student's t-test (*P<0.001; ** P<0.05; *** P<0.01; ns, non-statistically significant).

FIG. 4A-E: CART-2 shows increased mRNA delivery to splenic MZ B cells and NK cells in vivo compared to CART-1. (A) CART-2 and CART-1 each complexed to mRNA coding for the firefly luciferase reporter gene (Fluc) were administered intravenously (i.v.) to BALB/c mice. Whole-body bioluminescence was measured 6 hours later. (B) CART-2 or CART-1 complexed to Cy5-labeled Fluc mRNA were administered intravenously (i.v.) to BALB/c mice with 2 animals per group. After 2 hours the mice were sacrificed, and the spleens extracted for mechanical isolation of the total splenocytes. The cells were then stained for flow cytometry analysis. (C) Data shows average % of Cy5+ cells in total splenocytes of animals treated with CART-1 (green) and CART-2 (blue). Error bars show ±SD for 3 staining replicates. (D) Data shows average % of Cy5+ cells

in splenic B cells, myeloid cells, and T cells for animals treated with CART-1 (green) and CART-2 (blue). Error bars show ±SD for 3 staining replicates. (E) Data shows average % of Cy5+ cells in the splenic Marginal Zone B cells, Follicular B cells and NK cells for animals treated with CART-1 (green) and CART-2 (blue). Error bars show ±SD for 4 staining replicates. Statistical significance was calculated using an unpaired Student's t-test (*P<0.001; **P<0.05; ***P<0.01; ns, non-statistically significant).

FIG. 5A-B: CART-2 induces lymphopenia similarly to free FTY720. A. BALB/c mice were treated with either CART-1 or CART-2 formulated with Fluc mRNA. Mice treated with free FTY720 or naked Fluc mRNA were used as positive and negative control, respectively. All treatments were given i.v. Before treatment, blood was drawn from each mouse to calculate the baseline percentage of circulating T cells (CD3+) and B cells (B220+) among the total lymphocytes. One day after treatment mice were bled again to check for depletion of B and T cells from the peripheral blood. B. Trend over time of the average % of CD3+ (Left) and B220+ (Right) peripheral blood lymphocytes in the different treatment groups. Error bars show ±SD for 2 replicates per group. Statistical significance was calculated using an unpaired Student's t-test (*P<0.001; ** P<0.05; *** P<0.01; ns, non-statistically significant).

FIG. 6A-B: Graphical representation of the methods described herein for transfecting nucleic acids (mRNA) in vitro or in vivo into cells using receptor-mediated mRNA delivery with a small molecule functionalized CART. Depicted are CART nanoparticles functionalized with fingolimod and complexed with mRNA, which are then exposed to cells expressing the S1PR1 receptor, which internalize the CART nanoparticles where the mRNA cargo is released and expressed with high efficiency.

FIG. 7A-C: 1H NMR of protected CART-2, CART-3, and CART-4. A. 1H NMR of protected FTY720-A₁₂D₁₂ (CART-2) (CDCl₃, 400 MHZ) B. ¹H NMR of protected B-DMPA-A₁₁D₁₁ (CART-3) (CDCl₃, 400 MHz). C. ¹H NMR of protected PhDEA-A₁₄D₁₄ (CART-4) (CDCl₃, 400 MHz).

### DETAILED DESCRIPTION

The disclosure provides compositions and methods for nucleic acid delivery to mammalian cells in vitro and in vivo. Provided are compounds that are co-oligomers comprising non-linear branched lipophilic monomers (LP) and poly (alpha-aminoester) monomers (IM) covalently bound to a ligand moiety (A). In embodiments, the ligand moiety is selected for its binding to a cell surface receptor and therefore its ability to advantageously target the co-oligomer, and its nucleic acid cargo, to cells expressing the cell surface receptor. In embodiments, the ligand moiety may be a saccharide, a disaccharide, an oligosaccharide, a liposaccharide, a lipid, a peptide, an antibody, or a small organic molecule having binding specificity to a cell surface receptor. In the context of the present invention, a “small organic molecule” refers to a small organic compound, including heteroorganic and organometallic compounds, having a molecular weight less than about 1,000 grams per mole, or less than about 500 grams per mole and salts, esters, and other pharmaceutically acceptable forms of such compounds. In embodiments, the target cell surface receptor is a sphingosine 1-phosphate receptor 1 (S1PR1) and A is the small molecule fingolimod (FTY720). The copolymers described here may further comprise one or more optional linking groups covalently attached to one or both lipophilic monomers and linking the lipophilic monomer to the ligand moiety (A) or to an optional group, R^{2A}. For example, a co-oligomer according to the invention may comprise the following arrangement: A-L¹-[(LP¹)_{z1}-(IM)_{z2}-(LP²)_{z3}]_{z4}-L²-R^{2A}. However, other arrangements of the ligand moiety (A), and the optional linking and R^{2A} groups can be made. For example, R^{2A}-A-L¹-[(LP¹)_{z1}-(IM)_{z2}-(LP²)_{z3}]_{z4}-L²-R^{2A} or R^{2A}-L²-A-L¹-[(LP¹)_{z1}-(IM)_{z2}-(LP²)_{z3}]_{z4}-L²-R^{2A}, and the like. In particular embodiments, the disclosure provides co-oligomers comprising lipophilic monomers and degradable poly (alpha-aminoester) monomers in which a sphingosine-1-phosphate receptor modulator, fingolimod, is inserted into the oligomeric backbone and which, when complexed with nucleic acids, advantageously deliver their nucleic acid cargo to target B and T lymphocytes.

While various embodiments and aspects of the present disclosure are shown and described herein, it will be obvious to those skilled in the art that such embodiments and aspects are provided by way of example only. Numerous variations, changes, and substitutions will now occur to those skilled in the art without departing from the disclosure. It should be understood that various alternatives to the embodiments of the disclosure described herein may be employed in practicing the disclosure.

### Definitions

Unless the context indicates otherwise, it is specifically intended that the various features of the disclosure described herein can be used in any combination. Moreover, the disclosure also contemplates that in some embodiments any feature or combination of features set forth herein can be excluded or omitted. To illustrate, if the specification states that a complex has components A, B and C, it is specifically intended that any of A, B or C, or a combination thereof, can be omitted and disclaimed singularly or in any combination.

It must be noted that as used herein and in the appended claims, the singular forms “a”, “an”, and “the” include plural referents unless the context clearly dictates otherwise. Thus, for example, reference to “a cancer cell” includes a plurality of cancer cells. In other examples, reference to “a nucleic acid” or “nucleic acid” includes a plurality of nucleic acid molecules, i.e. nucleic acids.

The term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

Also as used herein, “and/or” refers to and encompasses any and all possible combinations of one or more of the associated listed items, as well as the lack of combinations when interpreted in the alternative (“or”).

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps “and those that do not materially affect the basic and novel characteristic(s)” of the recited embodiment. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the present disclosure.

The abbreviations used herein have their conventional meaning within the chemical and biological arts. The chemical structures and formulae set forth herein are constructed according to the standard rules of chemical valency known in the chemical sciences.

The terms “oligomer” and “polymer” are used interchangeably herein to refer to a compound that has a plurality of repeating subunits, which may be referred to as blocks or monomer units, or simply as monomers. The terms “co-oligomer” or “copolymer” are used interchangeably herein to refer to an oligomer or polymer that includes two or more different types of monomers. For example, in the context of the present invention the compounds provided are co-oligomers, which may also be referred to as copolymers, comprising at least two different types of monomers, a lipophilic block and a poly (alpha aminoester) block.

The term “polymerizable monomer” is used in accordance with its meaning in the art of polymer chemistry and refers to a compound that may covalently bind chemically to other monomer molecules (such as other polymerizable monomers that are the same or different) to form a polymer.

The term “block copolymer” is used in accordance with its ordinary meaning and refers to two or more portions (e.g., blocks) of polymerized monomers linked by a covalent bond. In embodiments, a block copolymer is a repeating pattern of polymers. In embodiments, the block copolymer includes two or more monomers in a periodic (e.g., repeating pattern) sequence. For example, a diblock copolymer has the formula: -B-B-B-B-B-B-A-A-A-A-A-, where ‘B’ is a first subunit and ‘A’ is a second subunit covalently bound together. A triblock copolymer therefore is a copolymer with three distinct blocks, two of which may be the same (e.g.,-A-A-A-A-A-B-B-B-B-B-B-A-A-A-A-A-) or all three are different (e.g., -A-A-A-A-A-B-B-B-B-B-B-C-C-C-C-C-) where ‘A’ is a first subunit, ‘B’ is a second subunit, and ‘C’ is a third subunit, covalently bound together. The term “random copolymer” refers to monomers randomly linked together in the polymer chain. The terms “random copolymer” and “statistical copolymer” are used interchangeably. The copolymers described herein may be block or random copolymers. For example, a diblock lipid could be a blocked or random mixture of two lipids. Similarly, a triblock copolymer comprising two lipid blocks and a cationic block could be blocked or random with respect to the sequence of monomer residues.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C1-C10 means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH—CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃, —CH₂—O—Si(CH₃)₃, R—S—S—R′, RO—S(O)x-OR′, and RO—P(O)x-OR′. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as-NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from an heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples off heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy) propyl, and the like).

The symbol “˜” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

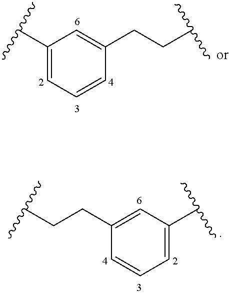 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —Cl₃, —CN, —CHO, —OH, —NH₂, —COOH, —CONH₂, —NO₂, —SH, —SO₂ CH₃—SO₃H, —OSO₃H, —SO₂NH₂, —NHNH₂, —ONH₂, —NHC(O)NHNH₂, substituted or unsubstituted C₁-C₅alkyl or substituted or unsubstituted 2 to 5 membered heteroalkyl). In embodiments, the alkylarylene is unsubstituted.

The term “alkylsulfonyl,” as used herein, means a moiety having the formula —S(O₂)—R′, where R′ is an alkyl group as defined above. R′ may have a specified number of carbons (e.g., “C₁-C₄ alkylsulfonyl”).

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)—NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O) R′, —S(O) 2R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In embodiments, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur(S), phosphorus (P), and silicon (Si).

A “substituent group,” as used herein, means a group selected from the following moieties:
(A) oxo, halogen, —CCl₃, —CBr₃, —CF₃, —Cl₃, CHCl₂, —CHBr₂, —CHF₂, —CHl₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHC₂, —OCHBr₂, —OCHI₂, —O CHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl, heteroalkyl, cycloalkyl, heterocycloalkyl, aryl, heteroaryl, substituted with at least one substituent selected from:
   (i) oxo, halogen, —CCl₃, —CBr₃, —CF₃, —Cl₃, CHCl₂, —CHBr₂, —CHF₂, —CHl₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHC₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl, heteroalkyl, cycloalkyl, heterocycloalkyl, aryl, heteroaryl, substituted with at least one substituent selected from:
      (a) oxo, halogen, —CCl₃, —CBr₃, —CF₃, —Cl₃, CHCl₂, —CHBr₂, —CHF₂, —CHl₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHC₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl, heteroalkyl, cycloalkyl, heterocycloalkyl, aryl, heteroaryl, substituted with at least one substituent selected from: oxo, halogen, —CCl₃, —CBr₃, —CF₃, —Cl₃, CHCl₂, —CHBr₂, —CHF₂, —CHl₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHC₂, —OCHBr₂, —OCHI₂, —OCHF₂, —OC H₂Cl, —OCH₂Br, —OCH₂I, —OCH₂F, —N₃, unsubstituted alkyl (e.g., C₁-C₈alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C6-C10 aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In embodiments of the compounds herein, each substituted or unsubstituted alkyl may be a substituted (e.g., substituted with a substituent group, a size-limited substituent group, or lower substituent group) or unsubstituted C₁-C₂₀ alkyl, each substituted or unsubstituted heteroalkyl is a substituted (e.g., substituted with a substituent group, a size-limited substituent group, or lower substituent group) or unsubstituted 2 to 20 membered heteroalkyl, each substituted or unsubstituted cycloalkyl is a substituted (e.g., substituted with a substituent group, a size-limited substituent group, or lower substituent group) or unsubstituted C₃-C₈ cycloalkyl, each substituted or unsubstituted heterocycloalkyl is a substituted (e.g., substituted with a substituent group, a size-limited substituent group, or lower substituent group) or unsubstituted 3 to 8 membered heterocycloalkyl, each or unsubstituted aryl is a substituted (e.g., substituted with a substituent group, a size-limited substituent group, or lower substituent group) or unsubstituted C₆-C₁₀ aryl, and/or each substituted or unsubstituted heteroaryl is a substituted (e.g., substituted with a substituent group, a size-limited substituent group, or lower substituent group) or unsubstituted 5 to 10 membered heteroaryl. In embodiments herein, each substituted or unsubstituted alkylene is a substituted (e.g., substituted with a substituent group, a size-limited substituent group, or lower substituent group) or unsubstituted C₁-C₂₀ alkylene, each substituted or unsubstituted heteroalkylene is a substituted (e.g., substituted with a substituent group, a size-limited substituent group, or lower substituent group) or unsubstituted 2 to 20 membered heteroalkylene, each substituted or unsubstituted cycloalkylene is a substituted (e.g., substituted with a substituent group, a size-limited substituent group, or lower substituent group) or unsubstituted C₃-C₈ cycloalkylene, each substituted or unsubstituted heterocycloalkylene is a substituted (e.g., substituted with a substituent group, a size-limited substituent group, or lower substituent group) or unsubstituted 3 to 8 membered heterocycloalkylene, each substituted or unsubstituted arylene is a substituted (e.g., substituted with a substituent group, a size-limited substituent group, or lower substituent group) or unsubstituted C₆-C₁₀ arylene, and/or each substituted or unsubstituted heteroarylene is a substituted (e.g., substituted with a substituent group, a size-limited substituent group, or lower substituent group) or unsubstituted 5 to 10 membered heteroarylene.

In embodiments, each substituted or unsubstituted alkyl is a substituted (e.g., substituted with a substituent group, a size-limited substituent group, or lower substituent group) or unsubstituted C₁-C₈alkyl, each substituted or unsubstituted heteroalkyl is a substituted (e.g., substituted with a substituent group, a size-limited substituent group, or lower substituent group) or unsubstituted 2 to 8 membered heteroalkyl, each substituted or unsubstituted cycloalkyl is a substituted (e.g., substituted with a substituent group, a size-limited substituent group, or lower substituent group) or unsubstituted C₃-C₇ cycloalkyl, each substituted or unsubstituted heterocycloalkyl is a substituted (e.g., substituted with a substituent group, a size-limited substituent group, or lower substituent group) or unsubstituted 3 to 7 membered heterocycloalkyl, each substituted or unsubstituted aryl is a substituted (e.g., substituted with a substituent group, a size-limited substituent group, or lower substituent group) or unsubstituted C₆-C₁₀ aryl, and/or each substituted or unsubstituted heteroaryl is a substituted (e.g., substituted with a substituent group, a size-limited substituent group, or lower substituent group) or unsubstituted 5 to 9 membered heteroaryl. In embodiments, each substituted or unsubstituted alkylene is a substituted (e.g., substituted with a substituent group, a size-limited substituent group, or lower substituent group) or unsubstituted C₁-C₈alkylene, each substituted or unsubstituted heteroalkylene is a substituted (e.g., substituted with a substituent group, a size-limited substituent group, or lower substituent group) or unsubstituted 2 to 8 membered heteroalkylene, each substituted or unsubstituted cycloalkylene is a substituted (e.g., substituted with a substituent group, a size-limited substituent group, or lower substituent group) or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted (e.g., substituted with a substituent group, a size-limited substituent group, or lower substituent group) or unsubstituted C₆-C₁₀ arylene, and/or each substituted or unsubstituted heteroarylene is a substituted (e.g., substituted with a substituent group, a size-limited substituent group, or lower substituent group) or unsubstituted 5 to 9 membered heteroarylene. In embodiments, the compound is a chemical species set forth in the Examples section, figures, or tables below.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Unless otherwise stated, structures depicted herein are also meant to include all stereochemical forms of the structure; i.e., the (R) and(S) configurations for each asymmetric center. Therefore, single stereochemical isomers as well as enantiomeric and diastereomeric mixtures of the present compounds, generally recognized as stable by those skilled in the art, are within the scope of the present disclosure.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

The term “nucleophilic moiety” refers to a chemical species or functional group that is capable of donating one or more electrons (e.g., 2) to an electrophile. In embodiments, a nucleophilic moiety refers to a chemical species or functional group that can donate an electron to an electrophile in a chemical reaction to form a bond.

The term “electrophilic moiety” refers to a chemical species or functional group that is capable of receiving one or more electrons (e.g., 2). In embodiments, an electrophilic moiety refers to a chemical species or functional group that has a vacant orbital and can thus accept one or two electrons to form a bond in a chemical reaction.

The term “oligoglycol moiety” refers to is a chemical entity with the general formula: R⁴⁰⁰—O—(CH2-CH2-O)n₃₀₀- [symbol] where R⁴⁰⁰ is H, substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl and n300 is an integer of 1 or more. In embodiments, R⁴⁰⁰ is H or alkyl.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY, 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this disclosure. The following definitions are provided to facilitate understanding of certain terms used frequently herein and are not meant to limit the scope of the present disclosure.

“Nucleic acid” refers to deoxyribonucleotides or ribonucleotides and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. messenger RNA (mRNA), small interference RNA (siRNA), short hairpin RNA (shRNA), micro RNA (miRNA), guide RNA (gRNA), CRISPR RNA (crRNA), transactivating RNA (tracrRNA), plasmid DNA (pDNA), minicircle DNA, genomic DNA (gNDA), and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids has one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine.; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism. An “inhibitory nucleic acid” is a nucleic acid (e.g. DNA, RNA, polymer of nucleotide analogs) that is capable of binding to a target nucleic acid (e.g. an mRNA translatable into a protein) and reducing transcription of the target nucleic acid (e.g. mRNA from DNA) or reducing the translation of the target nucleic acid (e.g. mRNA) or altering transcript splicing (e.g. single stranded morpholino oligo). In embodiments, the nucleic acid is RNA (e.g. mRNA). In embodiments the nucleic acid is 10 to 100,000 bases in length. In embodiments the nucleic acid is 50 and 10,000 bases in length. In embodiments the nucleic acid is 50 and 5,000 bases in length. In embodiments the nucleic acid is 50 and 1,000 bases in length.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. The terms apply to macrocyclic peptides, peptides that have been modified with non-peptide functionality, peptidomimetics, polyamides, and macrolactams. A “fusion protein” refers to a chimeric protein encoding two or more separate protein sequences that are recombinantly expressed as a single moiety.

The terms “peptidyl” and “peptidyl moiety” means a monovalent peptide.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Contacting” is used in accordance with its plain ordinary meaning and refers to the process of allowing at least two distinct species (e.g. chemical compounds including biomolecules or cells) to become sufficiently proximal to react, interact, associate, or physically touch. It should be appreciated, however, that the resulting reaction product can be produced directly from a reaction between the added reagents or from an intermediate from one or more of the added reagents which can be produced in the reaction mixture. In embodiments, contacting includes, for example, allowing a nucleic acid to interact with an endonuclease.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “cell” can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells.

The term “stem cell” or “stem cells” refers to a clonal, self-renewing cell population that is multipotent and thus can generate several differentiated cell types.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989, Molecular Cloning: A Laboratory Manual, 18.1-18.88).

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The term “plasmid” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

The term “exogenous” refers to a molecule or substance (e.g., nucleic acid or protein) that originates from outside a given cell or organism. Conversely, the term “endogenous” refers to a molecule or substance that is native to, or originates within, a given cell or organism.

A “vector” is a nucleic acid that is capable of transporting another nucleic acid into a cell. A vector is capable of directing expression of a protein or proteins encoded by one or more genes carried by the vector when it is present in the appropriate environment.

The term “codon-optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA. Such optimization includes replacing at least one, or more than one, or a significant number, of codons with one or more codons that are more frequently used in the genes of that organism. Given the large number of gene sequences available for a wide variety of animal, plant and microbial species, it is possible to calculate the relative frequencies of codon usage. Codon usage tables are readily available, for example, at the “Codon Usage Database” available at www.kazusa.or.jp/codon/. By utilizing the knowledge on codon usage or codon preference in each organism, one of ordinary skill in the art can apply the frequencies to any given polypeptide sequence, and produce a nucleic acid fragment of a codon-optimized coding region which encodes the polypeptide, but which uses codons optimal for a given species. Codon-optimized coding regions can be designed by various methods known to those skilled in the art.

A “cell culture” is an in vitro population of cells residing outside of an organism. The cell culture can be established from primary cells isolated from a cell bank or animal, or secondary cells that are derived from one of these sources and immortalized for long-term in vitro cultures.

The terms “transfection”, “transduction”, “transfecting” or “transducing” can be used interchangeably and are defined as a process of introducing a nucleic acid molecule and/or a protein to a cell. Nucleic acids may be introduced to a cell using non-viral or viral-based methods. The nucleic acid molecule can be a sequence encoding complete proteins or functional portions thereof. Typically, a nucleic acid vector, having the elements necessary for protein expression (e.g., a promoter, transcription start site, etc.). Non-viral methods of transfection include any appropriate method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. For viral-based methods, any useful viral vector can be used in the methods described herein. Examples of viral vectors include, but are not limited to retroviral, adenoviral, lentiviral and adeno-associated viral vectors. In some aspects, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

As used herein, the terms “specific binding” or “specifically binds” refer to two molecules forming a complex (e.g., a ribonucleoprotein and a transfection peptide) that is relatively stable under physiologic conditions.

Methods for determining whether a ligand binds another species (e.g., a protein or nucleic acid) and/or the affinity of such ligand-species interaction are known in the art. For example, the binding of a ligand to a protein can be detected and/or quantified using a variety of techniques such as, but not limited to, Western blot, dot blot, surface plasmon resonance method (e.g., BIAcore system; Pharmacia Biosensor AB, Uppsala, Sweden and Piscataway, N.J.), isothermal titration calorimetry (ITC), or enzyme-linked immunosorbent assays (ELISA).

Immunoassays which can be used to analyze immunospecific binding and cross-reactivity of the ligand include, but are not limited to, competitive and non-competitive assay systems using techniques such as Western blots, RIA, ELISA (enzyme linked immunosorbent assay), “sandwich” immunoassays, immunoprecipitation assays, immunodiffusion assays, agglutination assays, complement-fixation assays, immunoradiometric assays, and fluorescent immunoassays. Such assays are routine and well known in the art.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The terms “antigen” and “epitope” interchangeably refer to the portion of a molecule (e.g., a polypeptide) which is specifically recognized by a component of the immune system, e.g., an antibody, a T cell receptor, or other immune receptor such as a receptor on natural killer (NK) cells. As used herein, the term “antigen” encompasses antigenic epitopes and antigenic fragments thereof.

An exemplary immunoglobulin (antibody) structural unit can have a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “V_{H},” or “VH” refer to the variable region of an immunoglobulin heavy chain, including an Fv, scFv, dsFv or Fab; while the terms “variable light chain,” “V_{L}” or “VL” refer to the variable region of an immunoglobulin light chain, including an Fv, scFv, dsFv or Fab.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F (ab) 2′ and any combination of those or any other functional portion of an immunoglobulin peptide capable of binding to target antigen (see, e.g., FUNDAMENTAL IMMUNOLOGY (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

The term “co-oligomer complexed with a macromolecule” refers to a chemical complex formed with a co-oligomer as described herein and a macromolecule, such as a nucleic acid, bound to the co-oligomer wherein the complex is capable of being taken into a biological cell, such as a eukaryotic cell or prokaryotic cell, where the macromolecule then disassociates from the complex. In embodiments, the macromolecule is ionically bound to the co-oligomer. As used herein, the terms “cationic charge altering releasable transporter,” “CART” and the like refer to the co-oligomer portion of the complex as disclosed herein. The CART compounds are able to release the macromolecule component of the complex within the cell through the action of a poly (alpha-aminoester) monomer (alternatively, poly (alpha-aminoester) monomer) within the co-oligomer component, which undergoes an intramolecular rearrangement in response to intracellular pH, e.g., pH of about 7.4, thereby releasing the macromolecule, such as a nucleic acid, into the cell. In embodiments, the co-oligomer degrades rapidly within the cell (e.g. a half-life of less than 1 hour at pH 7.4). The co-oligomer complexed with macromolecule may be referred to as a polyplex, a complex, or an electrostatic complex, or more particularly, a CART/nucleic acid complex, a CART/oligonucleotide complex, or a CART/polynucleotide complex. In addition, the co-oligomer complexed with macromolecule, such as nucleic acid, may condense to form nanoparticles which serve to protect the nucleic acid cargo and may also further facilitate cellular entry. Such nanoparticles are generally in a size range of from 90-250 nanometers (nm) and more generally from about 50-300 nm.

The term “amphipathic polymer” as used herein refers to a polymer containing both hydrophilic and hydrophobic portions. In embodiments, the hydrophilic to hydrophobic portions are present in a 1 to 1 mass ratio. In embodiments, the hydrophilic to hydrophobic portions are present in a 1 to 2 mass ratio. In embodiments, the hydrophilic to hydrophobic portions are present in a 1 to 5 mass ratio. In embodiments, the hydrophilic to hydrophobic portions are present in a 2 to 1 mass ratio. In embodiments, the hydrophilic to hydrophobic portions are present in a 5 to 1 mass ratio. An amphipathic polymer may be a diblock or triblock copolymer. In embodiments, the amphiphilic polymer may include two hydrophilic portions (e.g., blocks) and one hydrophobic portion (e.g., block).

The term “non-linear branched lipophilic monomer” or the like, which may also be referred to as “lipid block”, refers to a region of the co-oligomer described herein comprising a lipophilic domain of non-linear, branched hydrocarbons as described herein.

The term “initiator” refers to a compound that is involved in a reaction synthesizing a co-oligomer having the purpose of initiating the polymerization reaction. Thus, the initiator is typically incorporated at the end of a synthesized polymer. For example, a plurality of molecules of one type (or formula) of monomer or more than one type of monomers (e.g. two different types of monomers) can be reacted with an initiator to provide a co-oligomer. The initiator can be present on at least one end of the resulting polymer and not constitute a repeating (or polymerized) unit(s) present in the polymer.

The terms “disease” or “condition” refer to a state of being or health status of a subject capable of being treated with a compound, pharmaceutical composition, or method provided herein. The disease can be an autoimmune, inflammatory, cancer, infectious, metabolic, developmental, cardiovascular, liver, intestinal, endocrine, neurological, or other disease. In embodiments, the disease is cancer (e.g. breast cancer, ovarian cancer, sarcoma, osteosarcoma, lung cancer, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, neuroblastoma).

The term “infection” or “infectious disease” refers to a disease or condition that can be caused by organisms such as a bacterium, virus, fungi or any other pathogenic microbial agents.

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g. hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

As defined herein, the term “inhibition”, “inhibit”, “inhibiting” and the like in reference to an activity and/or functionality of a molecule (e.g. polynucleotide or protein) means negatively affecting (e.g., decreasing or reducing) the activity or function of the molecule relative to the activity or function of the protein in the absence of the inhibition. Thus, inhibition includes, at least in part, partially or totally blocking stimulation, decreasing, preventing, or delaying activation, or inactivating, desensitizing, or down-regulating signal transduction or enzymatic activity or the amount of a protein or polynucleotide. Similarly an “inhibitor” is a compound that inhibits a target bio-molecule (i.e. nucleic acid, peptide, carbohydrate, lipid or any other molecules that can be found from nature), e.g., by binding, partially or totally blocking, decreasing, preventing, delaying, inactivating, desensitizing, or down-regulating activity of the target bio-molecule. In the context of disease prevention treatment, inhibition refers to reduction of a disease or symptoms of disease.

“Treatment,” “treating,” and “treat” are defined as acting upon a disease, disorder, or condition with an agent to reduce or ameliorate harmful or any other undesired effects of the disease, disorder, or condition and/or its symptoms. “Treating” or “treatment of” a condition or subject in need thereof refers to (1) taking steps to obtain beneficial or desired results, including clinical results such as an amelioration or reduction in one or more symptoms of the disease, disorder, or condition; (2) inhibiting the disease, for example, arresting or reducing the development or clinical progression of the disease, disorder, or condition, or any one or more of its clinical symptoms; (3) relieving the disease, for example, causing regression of the disease or its clinical symptoms; or (4) delaying or slowing disease progression.

The term “prevent,” “preventing” or “prevention”, in the context of a disease, refers to causing the clinical symptoms of the disease not to develop in a subject that does not yet experience or display symptoms of the disease. In embodiments, such prevention can be applied to a subject who can be considered predisposed of the disease, whereas in some other examples, the subject may not be necessarily considered predisposed to the disease.

As used herein, “administering” refers to the physical introduction of a composition to a subject, using any of the various methods and delivery systems known to those skilled in the art. Preferred routes of administration for the composition described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, the composition described herein can be administered via a non-parenteral route, such as a topical, epidermal or mucosal route of administration, for example, intranasally, orally, vaginally, rectally, sublingually or topically, for example to the skin, eye, or ear. Administering can also be performed, for example, once, a plurality of times, and/or over one or more extended periods.

The term “associated” or “associated with” in the context of a substance or substance activity or function associated with a disease means that the disease can be caused by (in whole or in part), or a symptom of the disease can be caused by (in whole or in part) the substance or substance activity or function. When the term is used in the context of a symptom, e.g. a symptom being associated with a disease or condition, it means that a symptom can be indicative of the disease or condition present in the subject who shows the symptom.

The term “subject,” “individual,” “host” or “subject in need thereof” refers to a living organism suffering from a disease or condition or having a possibility to have a disease or condition in the future. A term “patient” refers to a human that already has a disease or condition, e.g. a patient who has been diagnosed with a disease or condition or has one or more symptoms associated with a disease or condition. Non-limiting examples of subjects include humans, other mammals, bovines, rats, mice, dogs, monkeys, goat, sheep, cows, deer, and other non-mammalian animals.

The term “vaccine” refers to a composition that can provide active acquired immunity to and/or therapeutic effect (e.g. treatment) of a particular disease or a pathogen. A vaccine typically contains one or more agents that can induce an immune response in a subject against a pathogen or disease, i.e. a target pathogen or disease. The immunogenic agent stimulates the body's immune system to recognize the agent as a threat or indication of the presence of the target pathogen or disease, thereby inducing immunological memory so that the immune system can more easily recognize and destroy any of the pathogen on subsequent exposure. Vaccines can be prophylactic (e.g. preventing or ameliorating the effects of a future infection by any natural or pathogen, or of an anticipated occurrence of cancer in a predisposed subject) or therapeutic (e.g., treating cancer in a subject who has been diagnosed with the cancer). The administration of vaccines is referred to vaccination. In embodiments, a vaccine composition can provide nucleic acid, e.g. mRNA that encodes antigenic molecules (e.g. peptides) to a subject. The nucleic acid that is delivered via the vaccine composition in the subject can be expressed into antigenic molecules and allow the subject to acquire immunity against the antigenic molecules. In the context of the vaccination against infection disease, the vaccine composition can provide mRNA encoding antigenic molecules that are associated with a certain pathogen, e.g. one or more peptides that are known to be expressed in the pathogen (e.g. pathogenic bacterium or virus). In the context of cancer vaccine, the vaccine composition can provide mRNA encoding certain peptides that are associated with cancer, e.g. peptides that are substantially exclusively or highly expressed in cancer cells as compared to normal cells. The subject, after vaccination with the cancer vaccine composition, can have immunity against the peptides that are associated with cancer and kill the cancer cells with specificity.

The term “immune response” used herein encompasses, but is not limited to, an “adaptive immune response”, also known as an “acquired immune response” in which adaptive immunity elicits immunological memory after an initial response to a specific pathogen or a specific type of cells that is targeted by the immune response, and leads to an enhanced response to that target on subsequent encounters. The induction of immunological memory can provide the basis of vaccination.

The term “immunogenic” or “antigenic” refers to a compound or composition that induces an immune response, e.g., cytotoxic T lymphocyte (CTL) response, a B cell response (for example, production of antibodies that specifically bind the epitope), an NK cell response or any combinations thereof, when administered to an immunocompetent subject. Thus, an immunogenic or antigenic composition is a composition capable of eliciting an immune response in an immunocompetent subject. For example, an immunogenic or antigenic composition can include one or more immunogenic epitopes associated with a pathogen or a specific type of cells that is targeted by the immune response. In addition, an immunogenic composition can include isolated nucleic acid constructs (such as DNA or RNA) that encode one or more immunogenic epitopes of the antigenic polypeptide that can be used to express the epitope(s) (and thus be used to elicit an immune response against this polypeptide or a related polypeptide associated with the targeted pathogen or type of cells).

According to the methods provided herein, the subject can be administered an effective amount of one or more of agents, compositions or complexes, all of which are interchangeably used herein, (e.g. co-oligomer complexed with nucleic acid or vaccine composition) provided herein. The terms “effective amount” and “effective dosage” are used interchangeably. The term “effective amount” is defined as any amount necessary to produce a desired effect (e.g., transfection of nucleic acid into cells and exhibiting intended outcome of the transfected nucleic acid). Effective amounts and schedules for administering the agent can be determined empirically by one skilled in the art. The dosage ranges for administration are those large enough to produce the desired effects, e.g. transfection of nucleic acid, modulation in gene expression, gene-edition, induction of stem cells, induction of immune response and more. The dosage should not be so large as to cause substantial adverse side effects, such as unwanted cross-reactions, anaphylactic reactions, and the like. Generally, the dosage can vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary and can be administered in one or more dose administrations daily, for one or several days. Guidance can be found in the literature for appropriate dosages for given classes of pharmaceutical products. For example, for the given parameter, an effective amount can show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact dose and formulation can depend on the purpose of the treatment, and can be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)).

### Compounds

Provided are compounds that are co-oligomers comprising non-linear branched lipophilic monomers (LP) and poly (alpha-aminoester) monomers (IM) covalently bound to a ligand moiety (A).). The copolymers described here may further comprise one or more optional linking groups (L¹, L²) covalently attached to one or both lipophilic monomers, and/or to an optional group, R^{2A}. The arrangement of the optional groups and the ligand moiety around the copolymer core may be varied. For example, a co-oligomer according to the invention may comprise the following arrangement of groups: A-L¹-[(LP¹)_{z1}-(IM)_{z2}-(LP²)_{z3}]_{z4}-L²-R^{2A}, or R^{2A}-A-L¹-[(LP¹)_{z1}-(IM)_{z2}-(LP²)_{z3}]_{z4}-L²-R^{2A}, or R^{2A}-L²-A-L¹-[(LP¹)_{z1}-(IM)_{z2}-(LP²)_{z3}]_{z4}-L²-R^{2A}, and the like.

In an aspect, a co-oligomer has a structure of Formula (I) comprising a ligand moiety, A, a lipophilic polymer block, LP, and a poly (alpha aminoester) block, IM:

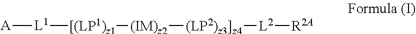

wherein:
   A is a ligand moiety that binds to a cell surface receptor, optionally A is selected from a saccharide, a disaccharide, an oligosaccharide, a liposaccharide, a lipid, a peptide, an antibody, and a small organic molecule;
   R^{2A} is hydrogen, halogen, —CCl₃, —CBr₃, —CF₃, —Cl₃, CHCl₂, —CHBr₂, —CHF₂, —CHl₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —O CHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   L¹ and L² are optional and each L¹ and L² is independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
   LP¹ and LP² are each independently a lipophilic polymer domain represented by

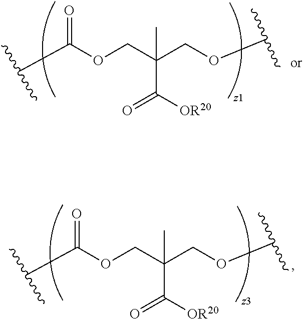

wherein
   z1 and z3 are independently from 1 to 100,
   wherein at least one of z1 or z3 is not 0; and
   R²⁰ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
IM is

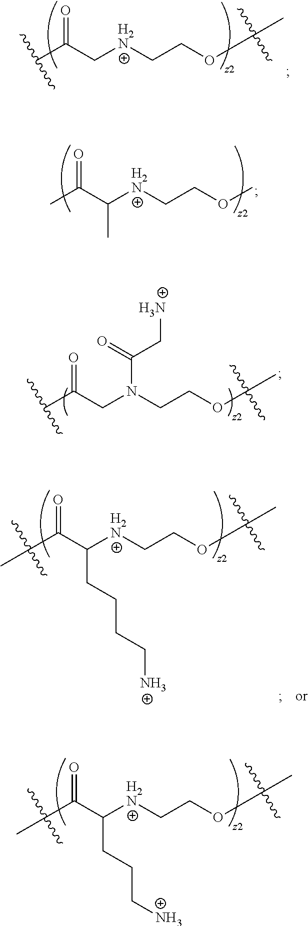

wherein z2 is from 2-100; and
z4 is 1 to 100.

In embodiments, the copolymer has a structure of Formula (Ia)

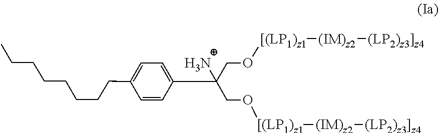

wherein LP¹, LP², IM, z1, z2, z3 and z4 are described herein.

In embodiments, L¹ is substituted or unsubstituted C₁-C₃ alkylene. In embodiments, L¹ is substituted or unsubstituted methylene. In embodiments, L¹ is substituted or unsubstituted C₁-C₆ alkylene, or substituted or unsubstituted 2 to 6 membered heteroalkylene. In embodiments, L¹ is substituted or unsubstituted C₁-C₃ alkylene, or substituted or unsubstituted 2 to 3 membered heteroalkylene.

In embodiments, L¹ is substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered). In embodiments, L¹ is unsubstituted alkylene, unsubstituted heteroalkylene, unsubstituted cycloalkylene, unsubstituted heterocycloalkylene, unsubstituted arylene, or unsubstituted heteroarylene. In embodiments, L¹ is unsubstituted alkylene (e.g., C₁-C₆ alkylene). In embodiments, L¹ is a bond.

In some embodiments, the co-oligomer complexed with nucleic acid has a co-oligomer having any of the foregoing formula in which L¹ is —CH₂—O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L¹ is —CH₂—O—.

In some embodiments, the co-oligomer can have any of the foregoing formula in which L¹ is —CH₂—O—,

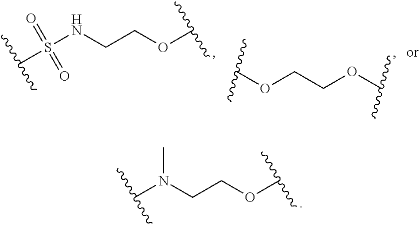

In embodiments, L¹ is —CH₂—O—. In embodiments, L¹ is

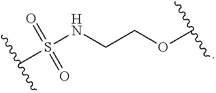 [SMILES: *OCCNS(*)(=O)=O]

In embodiments, L¹ is

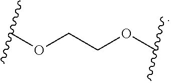 [SMILES: *OCCO*]

In embodiments, L¹ is

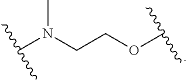 [SMILES: *OCCN(*)C]

In embodiments, L¹ is independently unsubstituted C₁-C₃ alkylene, -L²⁸-O—, or —O-L²⁸-, and L²⁸ is independently a bond or substituted or unsubstituted C₁-C₃ alkylene. In embodiments, L¹ is independently unsubstituted C₁-C₃ alkylene. In embodiments, L¹ is independently -L²⁸-O—. In embodiments, L¹ is independently —O-L²⁸-.

In embodiments, L²⁸ is independently a bond. In embodiments, L²⁸ is independently a substituted or unsubstituted C₁-C₃ alkylene. In embodiments, L²⁸ is independently a substituted C₁-C₃ alkylene. In embodiments, L²⁸ is independently an unsubstituted C₁-C₃ alkylene. In embodiments, L²⁸ is independently a substituted or unsubstituted methylene. In embodiments, L²⁸ is independently a substituted methylene. In embodiments, L²⁸ is independently an unsubstituted methylene. In embodiments, L²⁸ is independently a substituted or unsubstituted ethylene. In embodiments, L²⁸ is independently a substituted ethylene. In embodiments, L²⁸ is independently an unsubstituted ethylene. In embodiments, L²⁸ is independently a substituted or unsubstituted propylene. In embodiments, L²⁸ is independently a substituted propylene. In embodiments, L²⁸ is independently an unsubstituted propylene.

In embodiments, L² is substituted or unsubstituted C₁-C₃ alkylene. In embodiments, L² is substituted or unsubstituted methylene. In embodiments, L² is substituted or unsubstituted C₁-C₆ alkylene, or substituted or unsubstituted 2 to 6 membered heteroalkylene. In embodiments, L² is substituted or unsubstituted C₁-C₃ alkylene, or substituted or unsubstituted 2 to 3 membered heteroalkylene.

In embodiments, L² is substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C6-C10 or phenylene), or substituted or unsubstituted heteroarylene (e.g., 5 to 10 membered, 5 to 9 membered, or 5 to 6 membered). In embodiments, L² is substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene. In embodiments, L² is unsubstituted alkylene, unsubstituted heteroalkylene, unsubstituted cycloalkylene, unsubstituted heterocycloalkylene, unsubstituted arylene, or unsubstituted heteroarylene. In embodiments, L² is unsubstituted alkylene (e.g., C₁-C₆ alkylene). In embodiments, L² is a bond.

In embodiments, R^{2A} is independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R^{2A} is independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R^{2A} is independently unsubstituted alkyl, unsubstituted heteroalkyl, unsubstituted cycloalkyl, unsubstituted heterocycloalkyl, unsubstituted aryl, or unsubstituted heteroaryl. In embodiments, R^{2A} is independently hydrogen.

In embodiments, R^{2A} is independently hydrogen, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

In embodiments, R^{2A} is independently substituted or unsubstituted alkyl (e.g., C₁-C₈ alkyl, C₁-C₆ alkyl, or C₁-C₄ alkyl). In embodiments, R^{2A} is independently substituted alkyl (e.g., C₁-C₈ alkyl, C₁-C₆ alkyl, or C₁-C₄ alkyl). In embodiments, R^{2A} is independently an unsubstituted alkyl (e.g., C₁-C₈alkyl, C₁-C₆ alkyl, or C₁-C₄ alkyl). In embodiments, R^{2A} is independently substituted or unsubstituted heteroalkyl (e.g., 2 to 8 membered heteroalkyl, 2 to 6 membered heteroalkyl, or 2 to 4 membered heteroalkyl). In embodiments, R^{2A} is independently substituted heteroalkyl (e.g., 2 to 8 membered heteroalkyl, 2 to 6 membered heteroalkyl, or 2 to 4 membered heteroalkyl). In embodiments, R^{2A} is independently an unsubstituted heteroalkyl (e.g., 2 to 8 membered heteroalkyl, 2 to 6 membered heteroalkyl, or 2 to 4 membered heteroalkyl). In embodiments, R^{2A} is independently substituted or unsubstituted cycloalkyl (e.g., C₃-C₈ cycloalkyl, C₃-C₆ cycloalkyl, or C5-C6 cycloalkyl). In embodiments, R^{2A} is independently substituted cycloalkyl (e.g., C₃-C₈ cycloalkyl, C₃-C₆ cycloalkyl, or C5-C6 cycloalkyl). In embodiments, R^{2A} is an unsubstituted cycloalkyl (e.g., C₃-C₈ cycloalkyl, C₃-C₆ cycloalkyl, or C₅-C₆ cycloalkyl). In embodiments, R^{2A} is independently substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{2A} is independently substituted heterocycloalkyl (e.g., 3 to 8 membered heterocycloalkyl, 3 to 6 membered heterocycloalkyl, or 5 to 6 membered heterocycloalkyl). In embodiments, R^{2A} is independently an unsubstituted heterocycloalkyl (e.g., 3 to 8 membered heterocycloalkyl, 3 to 6 membered heterocycloalkyl, or 5 to 6 membered heterocycloalkyl). In embodiments, R^{2A} is independently substituted or unsubstituted aryl (e.g., C₆-C₁₀ aryl, C₁₀ aryl, or phenyl). In embodiments, R^{2A} is substituted aryl (e.g., C₆-C₁₀ aryl, C₁₀ aryl, or phenyl). In embodiments, R^{2A} is independently an unsubstituted aryl (e.g., C₆-C₁₀ aryl, C₁₀ aryl, or phenyl). In embodiments, R^{2A} is independently substituted or unsubstituted heteroaryl (e.g., 5 to 10 membered heteroaryl, 5 to 9 membered heteroaryl, or 5 to 6 membered heteroaryl). In embodiments, R^{2A} is independently substituted heteroaryl (e.g., 5 to 10 membered heteroaryl, 5 to 9 membered heteroaryl, or 5 to 6 membered heteroaryl). In embodiments, R^{2A} is independently an unsubstituted heteroaryl (e.g., 5 to 10 membered heteroaryl, 5 to 9 membered heteroaryl, or 5 to 6 membered heteroaryl).

In embodiments z2 is an integer from 2 to 90 (e.g. 5 to 90, 10 to 90 or 20 to 90), 2 to 80 (e.g. 5 to 80, 10 to 80 or 20 to 80), 2 to 70 (e.g. 5 to 70, 10 to 70 or 20 to 70), 2 to 50 (e.g. 5 to 50, 10 to 50 or 20 to 50) or 2 to 25 (e.g. 5 to 25, 10 to 25 or 20 to 25). In embodiments, z2 is 5 to 25. In embodiments, z2 is 10 to 25.

In embodiments, z1 and z3 are independently integers from 0 to 90 (e.g. 5 to 90, 10 to 90 or 20 to 90), 0 to 80 (e.g. 5 to 80, 10 to 80 or 20 to 80), 0 to 70 (e.g. 5 to 70, 10 to 70 or 20 to 70), 0 to 50 (e.g. 5 to 50, 10 to 50 or 20 to 50) or 2 to 25. In embodiments, z1 and z3 are independently integers from 2 to 90 (e.g. 5 to 90, 10 to 90 or 20 to 90), 2 to 80 (e.g. 5 to 80, 10 to 80 or 20 to 80), 2 to 70 (e.g. 5 to 70, 10 to 70 or 20 to 70), 2 to 50 (e.g. 5 to 50, 10 to 50 or 20 to 50) or 2 to 25 (e.g. 5 to 25, 10 to 25 or 20 to 25). In embodiments, z1 and z3 are independently integers from 5 to 30 (e.g., 5 to 25, or 5 to 20). In embodiments, z1 and z3 are independently integers from 5 to 30. In embodiments, z1 and z3 are independently 5 to 25. In embodiments, z1 and z3 are independently 5 to 20.

In embodiments, z4 is an integer from 1 to 100 (e.g. 5 to 100, 10 to 100 or 20 to 100), 1 to 90 (e.g. 5 to 90, 10 to 90 or 20 to 90), 1 to 80 (e.g. 5 to 80, 10 to 80 or 20 to 80), 1 to 70 (e.g. 5 to 70, 10 to 70 or 20 to 70), 1 to 50 (e.g. 5 to 50, 10 to 50 or 20 to 50) or 2 to 25. In embodiments, z4 is an integer from 2 to 90 (e.g. 5 to 90, 10 to 90 or 20 to 90), 2 to 80 (e.g. 5 to 80, 10 to 80 or 20 to 80), 2 to 70 (e.g. 5 to 70, 10 to 70 or 20 to 70), 2 to 50 (e.g. 5 to 50, 10 to 50 or 20 to 50) or 2 to 25 (e.g. 5 to 25, 10 to 25 or 20 to 25).

In embodiments, the pH-sensitive immolation domain has the formula:

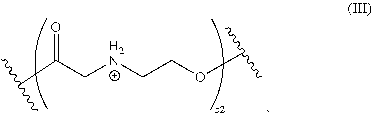

where z2 is from 1 to 100.

In embodiments, the lipophilic polymer domain has the formula:

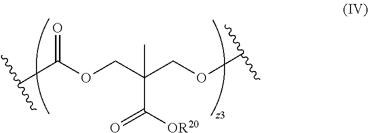

wherein
z2 is from 2 to 100;
R²⁰ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, R²⁰ is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R²⁰ is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R²⁰ is unsubstituted alkyl, unsubstituted heteroalkyl, unsubstituted cycloalkyl, unsubstituted heterocycloalkyl, unsubstituted aryl, or unsubstituted heteroaryl. In embodiments, R²⁰ is hydrogen or unsubstituted alkyl (e.g., C₁-C₆ alkyl). In embodiments, R²⁰ is hydrogen.

In embodiments, z2 is from 2 to 100. In embodiments, z2 is from 2 to 90. In embodiments, z2 is from 2 to 80. In embodiments, z2 is from 2 to 70. In embodiments, z2 is from 2 to 60. In embodiments, z2 is from 2 to 50. In embodiments, z2 is from 2 to 40. In embodiments, z2 is from 2 to 30. In embodiments, z2 is from 2 to 20. In embodiments, z2 is from or 2 to 10.

In embodiments, z2 is from 1 to 100. In embodiments, z2 is from 2 to 100. In embodiments, z2 is from 2 to 90. In embodiments, z2 is from 2 to 80. In embodiments, z2 is from 2 to 70. In embodiments, z2 is from 2 to 60. In embodiments, z2 is from 2 to 50. In embodiments, z2 is from 2 to 40. In embodiments, z2 is from 2 to 30. In embodiments, z2 is from 2 to 20. In embodiments, z2 is from 2 to 10.

In embodiments, the co-polymer has the structure of:

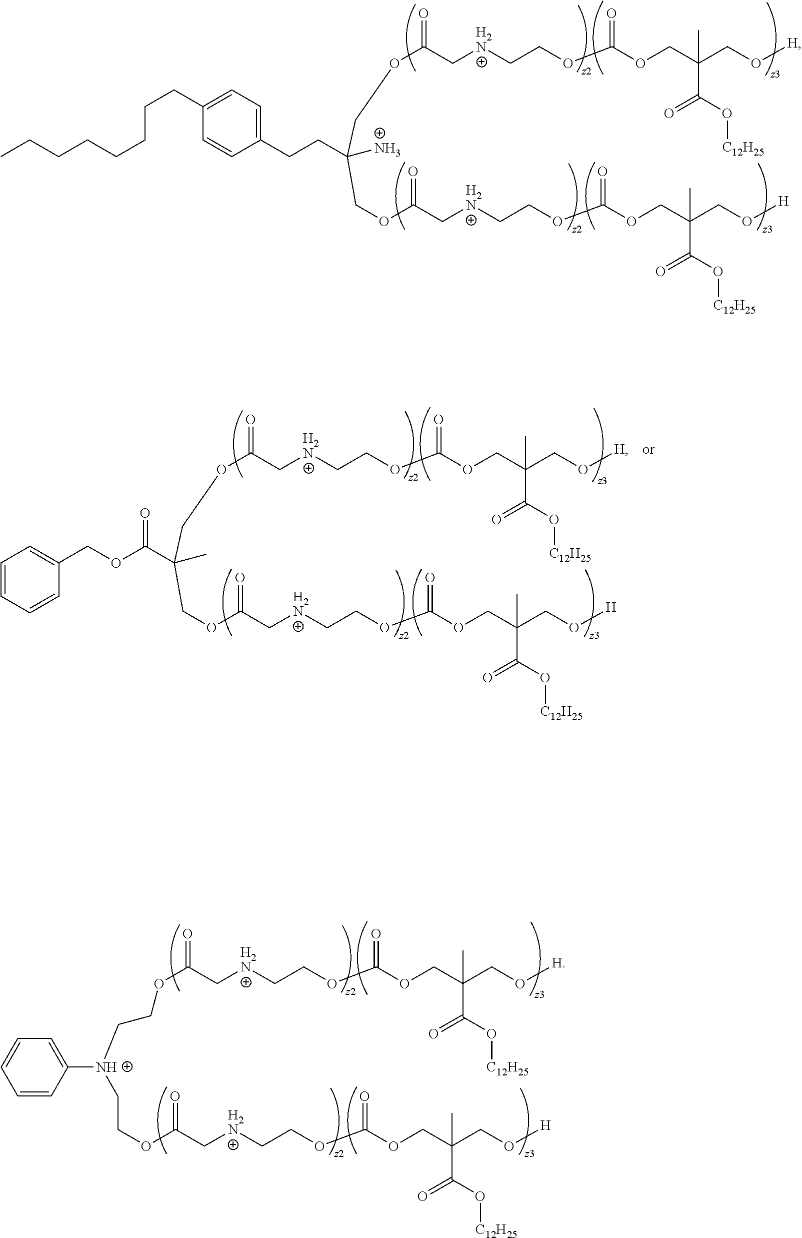

In embodiments, the co-polymer is

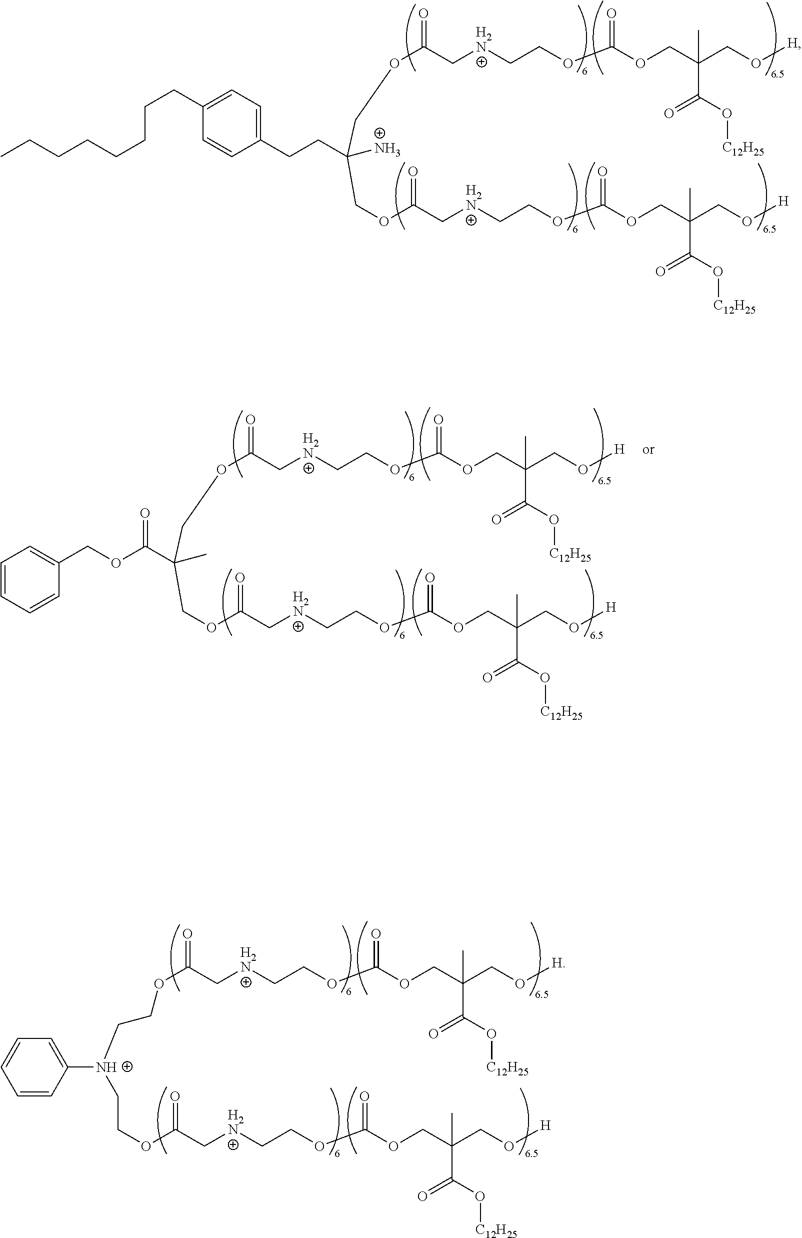

### Cell Penetrating Complexes

In a first aspect, there is provided a cell-penetrating complex including a nucleic acid non-covalently bound to a co-polymer, the co-polymer including a pH-sensitive immolation domain. In embodiments, one or more counter ions (e.g., anions) may also be present as countercharges to the positive charges in the co-polymer. In embodiments, the nucleic acid is non-covalently bound to the co-polymer. In embodiments, the nucleic acid is ionically bound to the co-polymer. In embodiments, the cell penetrating complex includes a plurality of optionally different nucleic acids (e.g. 1 to 10 additional nucleic acids, 1 to 5 additional nucleic acids, 1 to 5 additional nucleic acids, 2 additional nucleic acids or 1 additional nucleic acid). In embodiments, the nucleic acid is RNA. In embodiments, the nucleic acid is mRNA. In another aspect, the cell-penetrating complex may comprise any polyanionic molecule bound to a co-polymer as described herein, for example a polyphosphate, an inositol polyphosphate, a peptide nucleic acid (PNA), locked nucleic acid (LNA), or a protein having a polyanionic surface charge.

In embodiments, a ratio between the number of cations in the co-polymer molecules and the number of anions on the nucleic acid molecules present in a cell-penetrating complex can be about 1:1, about 5:1, about 10:1, about 20:1, about 30:1, about 40:1, about 50:1, about 60:1, about 70:1, about 80:1, about 90:1, about 10²:1, about 10³:1, about 10⁴:1, about 10⁵:1, about 10⁶:1, about 10⁷:1, about 10⁸:1, about 10⁹:1, about 10¹⁰:1, or more or any intervening ranges of the foregoing. In other embodiments, a ratio between the number of anions on the nucleic acid molecules and the number of cations on the co-polymer molecules present in a cell-penetrating complex can be about 1:1, about 5:1, about 10:1, about 20:1, about 30:1, about 40:1, about 50:1, about 60:1, about 70:1, about 80:1, about 90:1, about 10:1, about 102:1, about 103:1, about 104:1, about 105:1, about 106:1, about 107:1, about 108:1, about 109:1, about 1010:1, or more or any intervening ranges of the foregoing. In some preferred embodiments, this ratio is approximately 10 cationic charges on the amphipathic polymer molecule to 1 negative charge on the nucleic acid. Other embodiments can have 5 cationic charges on the amphipathic polymer molecule to 1 negative charge on the nucleic acid or 20 cationic charges on the amphipathic polymer molecule to 1 negative charge on the nucleic acid.

In embodiments, a ratio between the number of nucleic acid molecules and the number of co-polymer molecules present in a cell-penetrating complex can be about 1:1, about 10:1, about 10²:1, about 10³:1, about 10⁴:1, about 10⁵:1, about 10⁶:1, about 10⁷:1, about 10⁸:1, about 10⁹:1, about 10¹⁰:1, or more or any intervening ranges of the foregoing. In other embodiments, a ratio between the number of co-polymer molecules and the number of nucleic acid molecules present in a cell-penetrating complex can be about 1:1, about 10:1, about 10²:1, about 10³:1, about 10⁴:1, about 10⁵:1, about 10⁶:1, about 10⁷:1, about 10⁸:1, about 10⁹:1, about 10¹⁰:1, or more or any intervening ranges of the foregoing.

In one aspect, the disclosures herewith provide a cell-penetrating complex having a nucleic acid non-covalently bound to a copolymer having the structure of Formula (I):

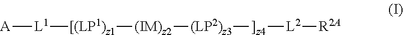

wherein A, L1, L², LP¹, LP², IM, z1, z2, z3 and z4 are as described herein.

In embodiments, the copolymer has a structure of Formula (Ia)

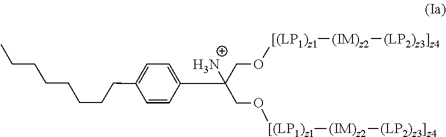

wherein LP¹, LP², IM, z1, z2, z3 and 74 are described herein.

Further to the cell-penetration complex disclosed herein and embodiments thereof, in embodiments, the nucleic acid may be DNA or RNA, such as messenger RNA (mRNA), small interference RNA (siRNA), short hairpin RNA (shRNA), micro RNA (miRNA), guide RNA (gRNA), CRISPR RNA (crRNA), transactivating RNA (tracrRNA), plasmid DNA (pDNA), minicircle DNA, genomic DNA (gNDA). The cell-penetration complex may further include a protein or peptide.

Further to the cell-penetration complex disclosed herein and embodiments thereof, in embodiments the cell-penetrating complex further includes a plurality of lipophilic moieties.

Further to the cell-penetration complex disclosed herein and embodiments thereof, in embodiments the cell-penetrating complex further includes a plurality of immolation domains.

Further to the cell-penetration complex disclosed herein and embodiments thereof, in embodiments, the counter-anion to the above cationic sequences can include common counterions known in the art, such as for example acetate, trifluoroacetate, triflate, chloride, bromide, sulfate, phosphate, succinate, or citrate. In embodiments, the counter-anion is acetate, trifluoroacetate, triflate, chloride, bromide, sulfate, phosphate, succinate, or citrate.

### Methods

The disclosure provides methods of targeted delivery of nucleic acids to particular types of cells and/or tissues in vitro, ex vivo, or in vivo. Any nucleic acid cargo may be ionically complexed to a co-oligomer as described herein for intracellular delivery and release. Accordingly, the disclosure provides methods of transfecting a nucleic acid into a target cell, the methods comprising contacting the target cell with a co-oligomer as disclosed herein complexed with the nucleic acid. In embodiments, the cell is a B or T lymphocyte.

In embodiments, the nucleic acid cargo is RNA or DNA. In embodiments, the RNA is messenger RNA (mRNA), small interference RNA (siRNA), short hairpin RNA (shRNA), micro RNA (miRNA), guide RNA (gRNA), CRISPR RNA (crRNA), or transactivating RNA (tracrRNA). In embodiments, the DNA is plasmid DNA (pDNA), minicircle DNA, or genomic DNA (gNDA).

In embodiments, the nucleic acid cargo is a therapeutic agent, or the nucleic acid encodes one or more therapeutic agents that upon intracellular delivery and release are transcribed into one or more therapeutic agents, such as cytokines or cellular receptors, for example a T cell receptor (TCR) or a chimeric antigen receptor (CAR). In embodiments, the CAR is a Lewis Y antigen CAR (i.e., a chimeric antigen receptor binding to Lewis Y antigen). In embodiments, the CAR is an anti-CD44v6 CAR. In embodiments, the CAR is an anti-NKG2D ligand CAR. In embodiments, the CAR is an anti-folate receptor beta CAR. In embodiments, the CAR is an anti-CD38 CAR. In embodiments, the CAR is an anti-CD20 CAR. In embodiments, the CAR is an anti-CD22 CAR. In embodiments, the CAR is an anti-FLT3 CAR. In embodiments, the CAR is an anti-CD7 CAR. In embodiments, the CAR is an anti-CD33 CAR. In embodiments, the CAR is an anti-CD123 CAR. In embodiments, the CAR is an anti-CLEC12A CAR.

The polyaminoester)s disclosed herein can be utilized as customizable, biodegradable, biocompatible materials for applications in biomedical therapies, imaging and devices. The copolymerization with biodegradable, non-toxic compounds materials such as valerolactone, caprolactone, lactide, and cyclic carbonates allows for tuning physical and biological properties including cargo release rates, hydrophobicity, incorporation of targeting ligands, biodistribution, and toxicity.

Accordingly, in some embodiments, the co-oligomers described here may be derived from cyclic amino-ester and cyclic methyl trimethylene carbonate (MTC) monomers. Cyclic amino-esters have the base structure of morpholin-2-one and homologs thereof, with multiple substitution patterns possible including the following.
(1) N-acylation with a variety of hydrophobic groups (e.g., R=alkyl, alkenyl, aryl, polycycles including steroids, heterocycles), cationic groups (e.g., ammonium, phosphonium, sulfonium, guanidinium, including acylation with amino acids such as glycine, lysine, ornithine, arginine), anionic groups (e.g., carboxylate, sulfate, phosphate), or hydrophilic (e.g., PEG) carbamates. Protection of the morpholine nitrogen with N-Boc or N-Cbz groups followed by organocatalytic ring opening oligomerization or polymerization can afford upon deprotection cationic polymer or oligomer backbones.
(2) Alpha-alkylation or functionalization next to the ester carbonyl with the aforementioned possible functionalities selected to allow for cargo complexation and subsequent cargo release by biodegradation.
(3) Alkylation proximal to the morpholine nitrogen with the aforementioned functionalities.
(4) A combination of the above modifications.

Additionally, copolymers or co-oligomers (block or statistical) can be made by mixing two or more morpholin-2-one monomers, or by the copolymerization (or co-oligomerization) of one or multiple morpholin-2-one monomers with one or multiple cyclic carbonate monomers described herein. These carbonate monomers can incorporate a similar variety of side chain functionality, notably lipophilic groups or cationic groups to modulate oligonucleotide stability, delivery, and release properties. Furthermore, a variety of other commercially available cyclic ester monomers can be used including but not limited to lactide, glycolide, valerolactone, and/or caprolactone to incorporate lipophilic functionality.

The synthesis of polyaminoesters and poly (carbonate-co-aminoester) s is achieved through the ring-opening polymerization and/or copolymerization of morpholine-2-one and cyclic carbonate monomers. The N-Boc protected morpholinone (MBoc) polymerizes to high conversion (>85%), tunable Mn (1 kDa-20 kDa), and low molecular weight distributions (Mw/Mn-1.1-1.3) using an organocatalytic system. Post-polymerization deprotection of the Boc groups affords a cationic (diprotic, secondary amine) water-soluble polymer (−0.5M in D20, stable for >3 days). Furthermore, copolymerization of MBoc with MTC-dodecyl carbonate monomers followed by deprotection give rise to moderately charged cationic materials in high yield (>60%) with narrow polydispersity <1.4 PDI) and tunable block length. Block length is controlled by the ratio of initiator to monomer.

The poly(aminoester) s described here are biocompatible and biodegradable. In certain embodiments, the poly(aminoester) s rapidly degrade through a unique pH-dependent intramolecular rearrangement to generate degradation products that are substantially nontoxic when the co-oligomer is administered in a therapeutic amount to a subject. In addition, the monomeric form, which is the expected product of further hydrolysis, can be used as a biomarker for phospholipid modification in the Maillard reaction. The carbonate segment of the poly(aminoester) degrades through hydrolysis and decarboxylation, and its byproducts have previously been shown to be non-toxic.

In embodiments, the methods described here may include complexation of the nucleic acid cargo with a co-oligomer in the presence of a coordinating metal such as Zn⁺², Mg⁺², Ca⁺²; a dynamic non-covalent cross linker such as a carbohydrate; a counterion such as Cl⁻, AcO⁻, succinate, or citrate; or a solubility modulator such as a lipid or a polyethyleneglycol (PEG), or any combination thereof.

In embodiments, a method of transfecting a nucleic acid into a cell as described herein may be part of a method for gene editing or genetic engineering. For example, one or more nucleic acids may be transfected using the co-oligomers described herein in a CRISPR-based system or a transposon-based system for gene editing or genetic engineering. In embodiments, gene editing may result in a DNA deletion, a gene disruption, a DNA insertion, a DNA inversion, a point mutation, a DNA replacement, a knock-in, or a knock-down. In one aspect, the nucleic acid transfected according to the methods described here may comprise one or more vectors having a first nucleotide sequence encoding a CRISPR-Cas system guide RNA that hybridizes with a target sequence in the genome of the cell and a second nucleotide sequence encoding a Cas9 protein. In certain embodiments, the first and second nucleotide sequence can be located on the same or different vectors. In some embodiments, the nucleic acid may comprise a CRISPR RNA (crRNA). In some embodiments, this crRNA can be in the same vector of the first nucleotide sequence encoding a CRISPR-Cas system guide RNA. In some embodiments, the nucleic acid may comprise a transactivating RNA (tracrRNA). In some embodiments, this tracrRNA can be in the same vector of the second nucleotide sequence encoding a Cas9 protein. In some embodiments, the Cas9 protein is codon optimized for expression in the transfected cell.

In another aspect, the nucleic acid may comprise one or more vectors having a first nucleotide sequence encoding a transposase and a second nucleotide sequence having a nucleic acid sequence of a gene of interest flanked by a transposase recognition site. In some embodiments, the first and second nucleotide sequences can be located on the same or different vectors. Transposase generally refers to an enzyme that can bind to a transposon and catalyze the movement of the transposon to another part of the genome by, e.g. a cut and paste mechanism or a replicative transposition mechanism. Introduction of transposase and a gene of interest flanked by a transposase recognition site in cells can induce insertion of the gene of interest into a cellular genome.

In some embodiments, a co-oligomer as described herein complexed with nucleic acid can be used as a vaccine. In some embodiments, a disease or condition that is targeted by the vaccine or vaccine composition can include, but not limited to, an autoimmune, inflammatory, cancer, infectious, metabolic, developmental, cardiovascular, liver, intestinal, endocrine, neurological, or other disease. In some embodiments, the nucleic acid that is contained in the vaccine or composition thereof can be a nucleic acid sequence encoding an antigenic or immunogenic epitope. In embodiments, two separate nucleic acids can encode two different immunogenic peptides. Therefore, in some embodiments, a vaccine composition described here can transfect (1) a first nucleic acid encoding a first immunogenic peptide that can induce more immediate treatment effect to an existing disease or condition and (2) a second nucleic acid encoding a different, second immunogenic peptide that is aimed to induce adaptive immunity in the subject for future occurrence of a different disease or condition. In some embodiments, the vaccine can deliver two or more different nucleic acids to a subject and each nucleic acid independently exhibits a therapeutic or prophylactic effect, respectively.

In embodiments a vaccine composition can have two or more different types of co-oligomer. Alternatively, a vaccine composition can have only a single type of co-oligomer. In some embodiments, a single type of co-oligomer can be non-covalently bound to one type (sequence) of nucleic acid. Alternatively, a single type of co-oligomer can be non-covalently bound to two or more types (sequences) of nucleic acid. Therefore in embodiments, a mixture of different types of co-oligomers, each of which is bound to a different sequence of nucleic acid, can be administered together to a subject in order to deliver two or more sequences (or types) of nucleic acids. Alternatively, a single type (or formula) of co-oligomer that is bound to multiple types (or sequences) of nucleic acid can be administered to a subject in order to deliver two or more sequences (or types) of nucleic acid. Still alternatively, a single type (or formula) of co-oligomer that is bound to a single sequence (or type) of nucleic acid can be administered to a subject.

In some embodiments, the nucleic acid that is contained the vaccine or composition thereof can be messenger RNA (mRNA), small interference RNA (siRNA), short hairpin RNA (shRNA), micro RNA (miRNA), guide RNA (gRNA), CRISPR RNA (crRNA), transactivating RNA (tracrRNA), plasmid DNA (pDNA), minicircle DNA, genomic DNA (gNDA). In alternative embodiments, the nucleic acid that is contained the vaccine or composition thereof can be mRNA. In some embodiments, nucleic acid is transfected into one or more cells in the subject via vaccination. In some embodiments, one or more than one nucleic acid sequences can be transfected via a vaccine composition. Therefore, in some embodiments, a vaccine composition contains two different nucleic acids, each of which encodes different antigenic peptides. Accordingly, when the vaccine is administered into a subject in need of the vaccination, two or more types of antigenic epitopes can be expressed and induce immune responses in the subject. In alternative embodiments, one type of nucleic acid can be transfected via vaccination such that one type of epitope can be expressed and induce an immune response in the subject.

In embodiments, the nucleic acid includes one or more vectors. In embodiments, the vector may include (a) a first polynucleotide encoding a CRISPR-Cas system guide RNA that hybridizes with a target sequence in the genome of the cell, and (b) a second polynucleotide encoding a Cas9 protein, optionally wherein the Cas9 protein is codon optimized for expression in the cell. In embodiments, the first (a) and second (b) polynucleotides are located in the same or different vectors.

In embodiments, the nucleic acid comprises a CRISPR RNA (crRNA), optionally wherein the crRNA is in the same vector as the first nucleotide sequence.

In embodiments, the nucleic acid comprises a transactivating RNA (tracrRNA). In embodiments, the tracrRNA is optionally in the same vector as the second nucleotide sequence.

In embodiments, the nucleic acid includes (a) a first polynucleotide encoding a transposase; and (b) a second polynucleotide comprising a nucleic acid sequence of a gene of interest flanked by a transposase recognition site. In embodiments, the first (a) and second (b) polynucleotides are located in the same or different vectors. In embodiments, the transposase recognizes and excises a genomic sequence of interest.

### Pharmaceutical Compositions

The disclosure also provides pharmaceutical compositions comprising a co-oligomer as described herein, which can be used for therapy. In some embodiments, the co-oligomer is complexed with a nucleic acid. In embodiments, the composition has a co-oligomer but not a cargo nucleic acid. In accordance with these embodiments, the cargo nucleic acid can be complexed with the co-oligomer before administration of the composition to a subject.

In some embodiments, a composition can be a vaccine or a composition thereof, i.e. a composition that contains the vaccine and optionally a pharmaceutically acceptable carrier. The vaccine or vaccine composition can be used to prevent and/or treat a disease or condition or a pathogen associated with the disease or condition. In some embodiments, the vaccine or vaccine composition contains a co-oligomer and a cargo nucleic acid. In some embodiments, the co-oligomer complexed with nucleic acid, when administered to a subject, can induce an immune response, i.e. immunogenic. This immunogenicity can be induced, at least in part, when one or more antigenic peptides encoded by the cargo nucleic acid are expressed in the transfected cells.

In some embodiments, pharmaceutical compositions may contain pharmaceutically acceptable excipients or additives depending on the route of administration. Examples of such excipients or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used can be chosen from, but not limited to, the above or combinations thereof, as appropriate, depending on the dosage form of the present disclosure.

In some embodiments, the pharmaceutically acceptable carrier is an immunological adjuvant. In embodiments, the immunological adjuvant can include, but is not limited to, agonists of Toll-like Receptors (TLRs), agonists of the STING pathway, agonistic antibodies against CD40, OX40, CTLA4, PD1, or PD1-L, Freund's adjuvant, bryostatins and ligands for CD40, OX40, CD137, PD1, CTLA4 and any combinations thereof. In some embodiments, the adjuvant can increase immunogenicity that is induced when a co-oligomer complexed with nucleic acid by co-administered with the complex to a subject.

Formulation of the pharmaceutical compositions of the present disclosure can vary according to the route of administration selected (e.g., solution, emulsion). Routes of administration can be, for example, intraperitoneal, intramuscular, subcutaneous, intravenous, intralymphatic, intraocular, topical skin, topical conjunctival, oral, intravesical (bladder), intrananal and intravaginal.

In some embodiments, the composition can include a cryoprotectant agent. Non-limiting examples of cryoprotectant agents include a glycol (e.g., ethylene glycol, propylene glycol, and glycerol), dimethyl sulfoxide (DMSO), formamide, sucrose, trehalose, dextrose, and any combinations thereof.

In some embodiments, the formulation is a controlled release formulation. The term “controlled release formulation” includes sustained release and time-release formulations. Controlled release formulations are well-known in the art. These include excipients that allow for sustained, periodic, pulse, or delayed release of the composition. Controlled release formulations include, without limitation, embedding of the composition into a matrix; enteric coatings; micro-encapsulation; gels and hydrogels; implants; and any other formulation that allows for controlled release of a composition.

In one aspect is provided a kit of parts having a co-oligomer complexed with nucleic acid or composition thereof. In another aspect is provided a kit of parts having a co-oligomer that is not bound to a nucleic acid or composition thereof. The kit can further contain a document or an instruction that describes a protocol for making a co-oligomer complexed with nucleic acid. The document or instruction of the kit can also describe a protocol for administering the composition to a subject in need thereof.

Therapeutic formulations described herein can be prepared for storage by mixing the active ingredients, i.e., immunogenic agent(s) having the desired degree of purity with optional physiologically acceptable carriers, excipients or stabilizers. Acceptable carriers, excipients, or stabilizers can be nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound (e.g., a second active agent in addition to the immunogenic agent(s) that has a co-oligomer complexed with nucleic acid), which may be selected for complementary activities that do not adversely affect each other. Such molecules can be suitably present in combination in amounts that can be effective for the purpose intended.

### Administration

In some aspects provided are methods for delivering a composition to cells or a subject so as to provide a desired activity into the cells or subject. In some embodiments, the composition can contain a co-oligomer complexed with nucleic acid where a cargo nucleic acid is non-covalently bound to a co-oligomer. The cargo nucleic acid, when transfected into the cells or administered to the subject, can provide a variety of intended effects, depending on the nature of the nucleic acid sequence. Some non-limiting examples of intended effects include modulation on gene expression, modulation of cellular pathways, genome-edition and induction of an immune response. In some embodiments, the composition can be administered to a subject in an effective amount that is sufficient to achieve at least part of the intended effects in the subject. In embodiments, the complexes described herein include a macromolecule that is a therapeutic such as a vaccine. In embodiments, the complexes may include a diagnostic agent, such as an MRI or CT contrast agent.

“Administration,” “administering” and the like, when used in connection with a composition refer both to direct administration, which may be administration to cells in vitro, administration to cells in vivo, administration to a subject by a medical professional or by self-administration by the subject and/or to indirect administration, which may be the act of prescribing a composition of the disclosure. When used herein in reference to a cell, refers to introducing a composition to the cell. Typically, an effective amount is administered, which amount can be determined by one of skill in the art. Any method of administration may be used. Compounds (e.g., drugs and antibodies) may be administered to the cells by, for example, addition of the compounds to the cell culture media or injection in vivo. Administration to a subject can be achieved by, for example, intravascular injection, direct intratumoral delivery, and the like.

Administering may mean oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the disclosure can be administered alone or can be co-administered to the patient. Co-administration is meant to include simultaneous or sequential administration of the compounds individually or in combination (more than one compound).

The dosage and frequency (single or multiple doses) administered to a subject can vary depending upon a variety of factors, for example, whether the subject suffers from another disease, its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and compositions described herein including embodiments thereof. Adjustment and manipulation of established dosages (e.g., frequency and duration) are well within the ability of those skilled in the art.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

In some embodiments, the subject is a mammal, for example a human, a non-human primate, a murine (i.e., mouse and rat), a canine, a feline, or an equine. In embodiments, the subject is a human.

In some embodiments, a composition can be administered in a dose (or an amount) of about 1 ng/kg of subject body weight, about 10 ng/kg of subject body weight, about 50 ng/kg of subject body weight, about 100 ng/kg of subject body weight, about 500 ng/kg of subject body weight, about 1 μg/kg of subject body weight, about 10 μg/kg of subject body weight, about 50 μg/kg of subject body weight, about 100 μg/kg of subject body weight, about 150 μg/kg of subject body weight, about 200 μg/kg of subject body weight, about 250 μg/kg of subject body weight, about 300 μg/kg of subject body weight, about 350 μg/kg of subject body weight, about 375 μg/kg of subject body weight, about 400 μg/kg of subject body weight, about 450 μg/kg of subject body weight, about 500 μg/kg of subject body weight, about 550 μg/kg of subject body weight, about 600 μg/kg of subject body weight, about 650 μg/kg of subject body weight, about 700 μg/kg of subject body weight, about 750 μg/kg of subject body weight, about 800 μg/kg of subject body weight, about 850 μg/kg of subject body weight, about 900 μg/kg of subject body weight, about 1 mg/kg of subject body weight, about 10 mg/kg of subject body weight, about 50 mg/kg of subject body weight, about 100 mg/kg of subject body weight, about 500 mg/kg of subject body weight, about 1 g/kg of subject body weight or more or any intervening ranges of the of the foregoing. In some embodiments, a composition can be administered in a dose (or an amount) of about 0.5 μg, about 1.0 μg, about 1.5 μg, about 2.0 μg, about 2.5 μg, about 3.0 μg, about 3.5 μg, about 4.0 μg, about 4.5 μg about 5.0 μg, about 5.5 μg, about 6.0 μg, about 6.5 μg, about 7.0 μg, about 7.5 μg, about 8.0 μg, about 8.5 μg, about 9.0 μg, about 9.5 μg, about 1.0 mg, about 1.5 mg, about 2.0 mg, about 2.5 mg, about 3.0 mg, about 3.5 mg, about 4.0 mg, about 4.5 mg about 5.0 mg, about 5.5 mg, about 6.0 mg, about 6.5 mg, about 7.0 mg, about 7.5 mg, about 8.0 mg, about 8.5 mg, about 9.0 mg, about 9.5 mg, about 1 g or more or any intervening ranges of the foregoing. In some embodiments, a composition can be administered in a dose (or an amount) of about 7.5 μg or about 0.375 mg/kg of subject body weight. Administration can be repeated over a desired period, e.g., repeated over a period of about 1 day to about 5 days or once every several days, for example, about five days, over about 1 month, about 2 months, etc. The weight herein can be a weight of a co-oligomer complexed with nucleic acid or a weight of a composition or pharmaceutical formulation thereof.

In embodiments, a composition can be administered intravenously, subcutaneously or intratumorally. In embodiments, a formulation or a pharmaceutical composition can be administered intravenously, subcutaneously or intratumorally.

In embodiments, a composition can be administered systemically or locally (e.g. intratumoral injection, intravenous injection) at intervals of 6 hours, 12 hours, daily or every other day or on a weekly or monthly basis to elicit the desired benefit or otherwise provide a therapeutic effect.

In embodiments, a response rate to a composition, in particular a cancer vaccine, can be reduced as compared to baseline reference or control reference. The term “response rate” is used herein in its customary sense to indicate the percentage of patients who respond with cancer recession following treatment. Response rates include, for example, partial or complete recession. A partial response includes an about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 97%, about 98%, or about 99% recession of cancer cells. In some embodiments, the control reference is obtained from a healthy subject, a cancer subject (e.g., the cancer subject being treated or another cancer subject), or any population thereof.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

Charge-altering releasable transporters (CARTs) are mRNA delivery vehicles showed to be effective platforms for mRNA cancer vaccines and transfection of lymphocytes in vitro and in vivo. Nevertheless, despite recent advancements, transfection of T and B cells still poses significant challenges. Here we exploited the chemical versatility of CARTs to conjugate the small molecule drug fingolimod (FTY720) to the CART core structure. Fingolimod binds to the sphingosine 1-phosphate receptor 1 (S1PR1), highly expressed on lymphocytes. Our goal was to increase mRNA delivery and expression in lymphocytes through a specific ligand-receptor interaction. Compared to non-fingolimod-conjugated analogue, the fingolimod-conjugated CART achieved superior transfection of activated human and murine T and B lymphocytes in vitro. Also, the fingolimod-conjugated CART showed higher transfection only in S1PR1+ expressing cells and this advantage was lost when cells were exposed to free fingolimod before transfection. In vivo, the fingolimod-conjugated CART showed increased mRNA delivery to the murine spleen with a predilection for B cells, particularly Marginal Zone B cells, and NK cells while inducing peripheral blood T cell depletion similarly to free fingolimod. Thus, we showed that functionalization of CARTs with a small molecule drug can increase transfection of a cellular population of interest while conferring some of the pharmacological properties of the conjugated small molecule to the CARTs.

During the last decade, therapeutics based on in vitro transcribed (IVT) mRNA have de facto become a new class of drugs with a wide variety of applications ranging from cancer immunotherapy to protein replacement therapy, genome editing and vaccine development. However, the clinical translation and application of mRNA-based therapies is hampered by the in vivo stability of mRNA which requires delivery vehicles (DVs) capable of efficiently protecting and releasing the cargo mRNA into the cytoplasm of target cells with minimal cytotoxicity. Currently, the most used mRNA DVs for both in vivo and in vitro applications are lipid nanoparticles (LNPs) and cationic polymers, which offer less immunogenicity and less cytotoxicity compared to respectively viral vectors and physical methods like electroporation. However, current non-viral mRNA DVs offer significant limits in terms of cost, formulation, stability, toxicity, and target specificity. To overcome these challenges, we have previously described the synthesis of biodegradable mRNA DVs named charge-altering releasable transporters (CARTs). These materials are readily produced using an established organo-catalytic ring-opening polymerization strategy and can efficiently protect and deliver mRNA to cells both in vivo and in vitro with minimal cytotoxicity.

Furthermore, we have previously demonstrated the utility of CARTs in the development of a SARS-Cov-2 mRNA vaccine as well as in several cancer immunotherapy strategies. Specifically, we have used CARTs for the in vivo delivery of mRNA coding either for an antigen (vaccination) or effector molecules (gene therapy/in situ vaccination) and with these approaches we were able to cure mice with tumors in multiple cancer models. We also showed that the chemistry of CARTs can be manipulated to increase delivery and expression of the cargo mRNA into lymphocytes both in vitro and in vivo. Historically, the efficient transfection of lymphocytes has been limited by significant cytotoxicity, inefficient gene delivery, inefficient expression of the delivered gene, expression associated with nonviral DVs, and by the immunogenicity and mutagenic potential of viral vectors.

While significant advancements have been made recently, including the development of new polycationic polymeric DVs, in vitro and in vivo delivery of mRNA to lymphocytes remains an important, but unsolved challenge in gene therapy with potential applications not only in cancer immunotherapy but also in autoimmunity and infectious disease. To address this issue, here we describe the synthesis and characterization of a novel mRNA DV named FTY720-CART.

FTY720 (fingolimod) is a small molecule drug, FDA-approved for treatment of patients with Multiple Sclerosis. FTY720 acts as a superagonist for the sphingosine 1 phosphate receptor 1 (S1PR1), highly expressed on T cells and B cells in secondary lymphoid organs, causing internalization of the receptor and decreased efflux of lymphocytes to the bloodstream leading to significant lymphopenia which is at least in part responsible for the immunosuppressive effects of FTY720 in Multiple Sclerosis.

We were inspired by this mechanism to synthesize CARTs containing FTY720 as an S1PR1-targeting moiety. Our goal was to create an mRNA DV that can be efficiently directed to T cells and B cells through a specific receptor-ligand interaction with the aim of facilitating transfection of lymphocytes both in vitro and in vivo for therapeutic applications. Herein, we report a new class of CARTs incorporating FTY720 as an integral structural component of the CART as a strategy to enhance the in vivo delivery of mRNA to T cells and B cells by exploiting the specific FTY720-S1PR1 receptor-ligand interaction. We demonstrate that CARTs incorporating FTY720 show enhanced transfection in cells expressing the S1PR1 receptor both in cell culture and in vivo, and that this enhanced transfection is abrogated upon pre-treatment with FTY720 in vitro. The FTY720-CARTs exhibit enhanced in vivo mRNA delivery to B cell and NK cells in the spleen upon IV injection, relative to CARTs lacking FTY720. Moreover, the FTY720-CARTs, when administered IV, induce peripheral T-cell depletion analogous to that exhibited by FTY720 administration.

### CART Synthesis and Characterization

CART-1 (Bn-D₁₃A₁₁), the N-boc morpholinone and MTC-dodecyl monomers were synthesized as previously reported (Mn(GPC)=6000 Da, Ð=1.25).¹⁷ CART-2 (FTY720-D₁₂A₁₂) was prepared by a slight modification of this procedure. In a nitrogen-filled glovebox, N-Boc morpholinone (21.5 mg, 0.1 mmol, 10.9 equiv) is dissolved in toluene (50 μL). To this solution is added thiourea catalyst (TU, 1.8 mg, 0.005 mmol, 0.5 equiv), diazabycycloundecene (DBU, 0.78 mg, 0.005 mmol, 0.5 equiv) and N-Boc protected FTY720 initiator (4 mg, 0.01 mmol, 1 equiv) as a solution in 50 μL toluene. The reaction was stirred for 2.5 hours, then MTC-dodecyl was added as a solid (35 mg, 0.1 mmol, 10.9 equiv). The reaction mixture was allowed to stir for an additional 1.5 hours. The reaction is removed from the glovebox and quenched with 100 uL of acetic acid. The crude mixture is dialyzed overnight in (DCM/MeOH, 3.5 kDa M.W, cut-off). An aliquot of the protected material is subjected to size-exclusion gel permeation chromatography (Mn(GPC)=9700 Da, Ð=1.20). Concentration after dialysis afforded 21 mg clear residue which was deprotected with 10% TFA in dry DCM (v/v, 2.1 mL, stirred for 4 hours at RT, collected by rotary evaporation). End group analysis of the protected polymer by ¹H NMR showed 12 dodecyl carbonate units and 12 cationic aminoester units per FTY720 initiator. ¹H NMR (400 MHz, Chloroform-d) δ: 7.07 (s, 4H), 4.5-4.2 (br, 68H), 4.1 (m, 24H), 4.03-3.89 (br, 24H), 3.59-3.46 (br, 24H), 1.68-1.53 (br, 26H), 1.49-1.36 (m, 126H), 1.35-1.18 (br, 234H), 0.86 (m, 36H).

CART-3 and CART-4 were prepared by the same method as CART-2 with the substitution of benzyl-2,2-bis(hydroxymethyl) propionate (B-DMPA) or N-phenyldiethanolamine (PhDEA) in place of the N-Boc protected FTY720 initiator respectively. Additional details are provided in the SI. CART-3: ¹H NMR (400 MHZ, Chloroform-d) δ: 7.40-7.30 (br, 5H), 5.18-5.13 (br, 2H), 4.5-4.16 (br, 69H), 4.11 (m, 22H), 4.04-3.89 (m, 22H), 3.60-3.44 (m, 22H), 1.69-1.56 (m, 27H), 1.49-1.37 (br, 106H), 1.36-1.16 (br, 239H), 0.87 (m, 33H); CART-4: ¹H NMR (400 MHZ, Chloroform-d) δ: 7.22 (m, 2H), 6.74 (m, 3H), 4.5-4.15 (br, 79H), 4.1 (m, 28H), 4.03-3.86 (br, 30H), 3.59-3.41 (br, 30H), 1.68-1.51 (m, 34H), 1.42 (d, 124H), 1.36-1.15 (br, 286H), 0.86 (m, 42H).

CART with saccharide targeting ligand, Method A: In a nitrogen-filled glovebox, (1R,8S,9s)-Bicyclo[6.1.0]non-4-yn-9-ylmethanol (1 equiv) and MTC-dodecyl (10 equiv) are stirred in DCM (50 μL) until dissolved. To this solution is added thiourea catalyst (0.6 equiv) and diazabycycloundecene (0.6 equiv) as a solution in 50 uL DCM. The reaction was stirred for 1.5 hours, then N-Boc morpholinone (10 equiv) was added as a solid. The reaction mixture was allowed to stir for an additional 2.5 hours. The reaction is removed from the glovebox and quenched with 100 μL of acetic acid. The crude mixture is dialyzed overnight in (DCM/MeOH, 3.5 kDa M.W, cut-off). The resulting CART polymer is dissolved in chloroform and mixed with a desired azido ligand (in this case 1-Azido-1-deoxy-β-D-glucopyranoside) dissolved in DMSO, this mixture is stirred for 12 hours. The crude mixture is dialyzed overnight in (DCM/MeOH, 3.5 kDa M.W, cut-off). The resulting material was deprotected with 10% TFA in dry DCM (v/v, 5 mL, stirred for 4 hours at RT, collected by rotary evaporation).

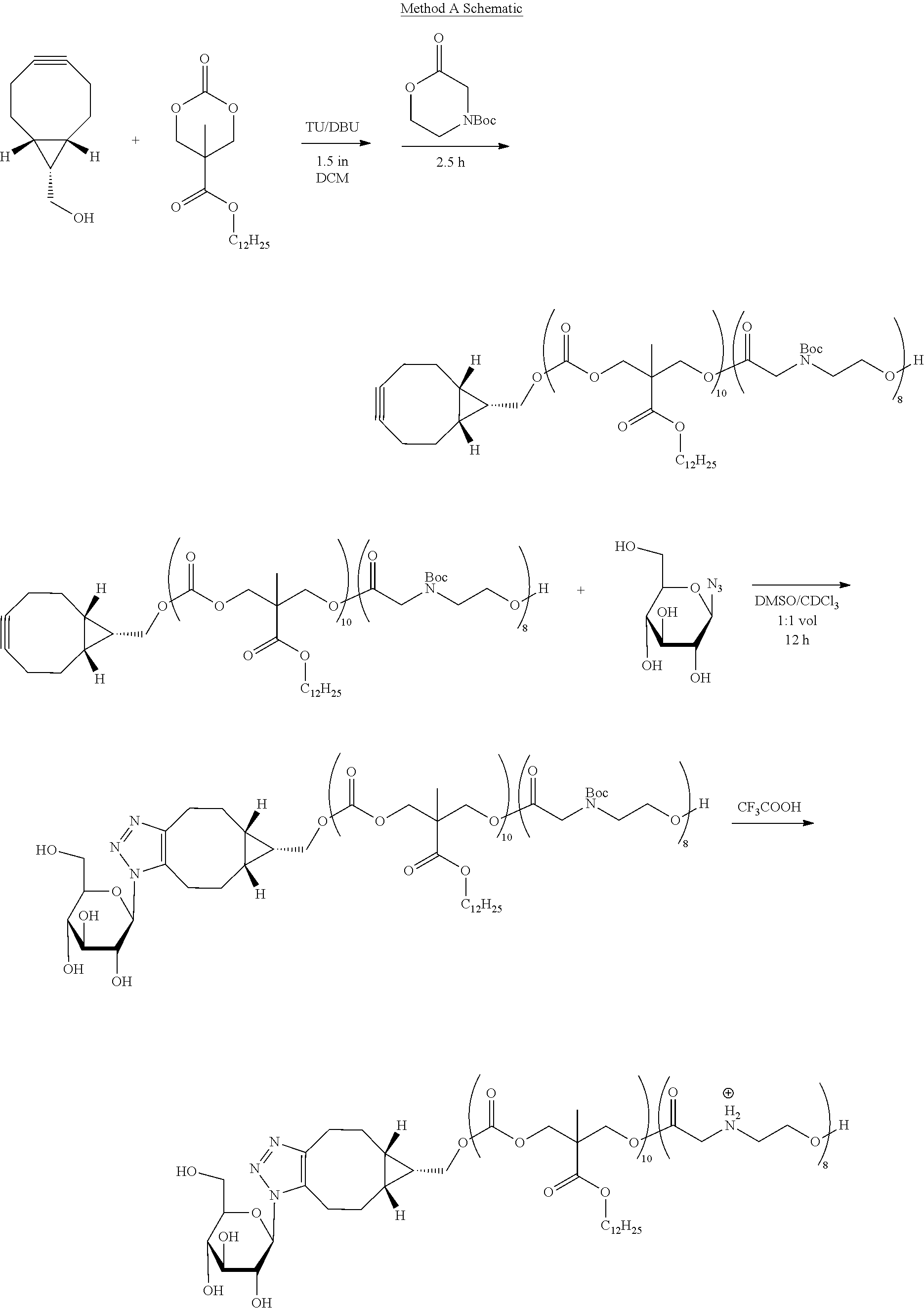

CART with saccharide targeting ligand, Method B: In a nitrogen-filled glovebox, 1,2:3,4-Di-O-isopropylidene-α-D-galactopyranose (1 equiv) and MTC-dodecyl (10 equiv) are stirred in DCM (50 μL) until dissolved. To this solution is added thiourea catalyst (0.6 equiv) and diazabycycloundecene (0.6 equiv) as a solution in 50 uL DCM. The reaction was stirred for 1.5 hours, then N-Boc morpholinone (10 equiv) was added as a solid. The reaction mixture was allowed to stir for an additional 2.5 hours. The reaction is removed from the glovebox and quenched with 100 μL of acetic acid. The crude mixture is dialyzed overnight in (DCM/MeOH, 3.5 kDa M.W, cut-off). The resulting material was deprotected with 10% TFA in dry DCM (v/v, 5 mL, stirred for 4 hours at RT, collected by rotary evaporation).

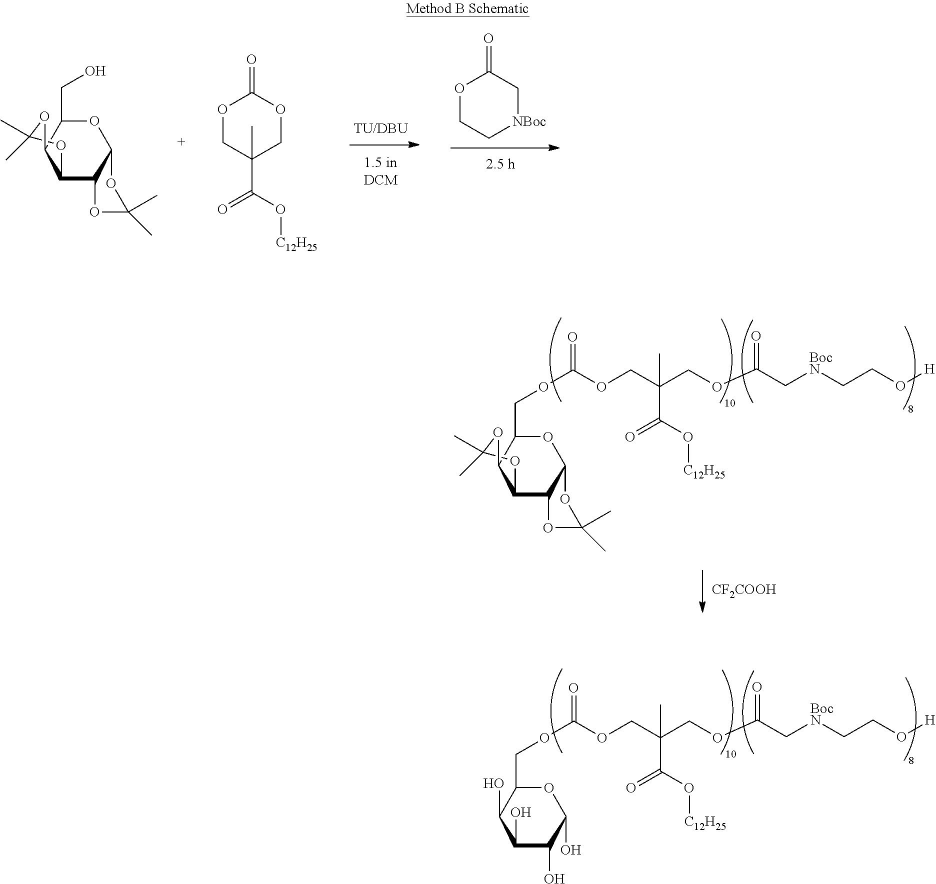

### Mice and Cells Lines

Six-to-eight-week-old female BALB/c mice were purchased from The Charles River Laboratory and housed in the Laboratory Animal Facility of the Stanford University Medical Center (Stanford, CA). All experiments were approved by the Stanford Administrative Panel on Laboratory Animal Care and conducted in accordance with Stanford University Animal Facility and NIH guidelines. Jurkat cells were obtained from ATCC (ATCC no. TIB-152). This is a human T lymphoblastic cell line derived from the peripheral blood of a 14-year-old male with acute T lymphoblastic leukemia. The K562 cell line was obtained from ATCC (ATCC no. CCL-243). These cells were generated from the pleural effusion of a 53-year-old female with chronic myelogenous leukemia in terminal blast crisis. CHO cells (Chinese Hamster Ovary) were obtained from the Levy lab inventory. Mycoplasma testing of cell lines was performed with 3-month intervals using a MycoAlert Detection Kit (Lonza). Jurkat and K562 cells were maintained in RPMI medium 1640 with L-Glutamine (cellgro; Cornig) supplemented with 10% heat-inactivated FCS (HyClone), 1% penicillin/streptomycin (Gibco) and 50 uM of 2-mercaptoethanol (Gibco) to obtain the complete medium. CHO cells were maintained in ATCC formulated F-12K medium supplemented with 10% heat-inactivated FCS (HyClone), 1% penicillin/streptomycin (Gibco) and 50 uM of 2-mercaptoethanol (Gibco). All cell cultures were grown at 37° C. in a 5% CO₂ atmosphere. Cells were passaged at ˜80% confluence and all experiments were performed within five cell passages after thawing with 0.05% trypsin-EDTA used for CHO cells.

### Isolation and In Vitro Activation of Splenic Murine B and T Cells

Single cell suspensions were prepared from the spleens of 6- to 8-weeks old female BALB/c mice through mechanical dissociation using a 70-μm cell strainer (BD Biosciences). The cells were recovered in ice-cold serum-free RPMI media, after which the sample was centrifuged, and the pellet resuspended in ACK buffer for 10 minutes to lyse any red blood cells present. The obtained splenocytes were then washed twice with PBS and finally recovered in 10% FCS RPMI. Where lymphocytes activation was not required, the cells were rested in 10% FCS RPMI for 12 hours before transfection. Where lymphocytes activation was required, we cultured the total splenocytes in 10% FCS RPMI media supplemented with an anti-CD28 monoclonal antibody (mAb) at 0.5 μg/mL (BD Pharmingen, hamster anti-mouse NA/LE) and an anti-CD3 mAb at 0.05 μg/mL (BD Pharmingen, hamster anti-mouse NA/LE) for 24 hours at 37° C. in a 5% CO2 atmosphere. This resulted in direct stimulation of T cells with subsequent cytokine production and bystander activation of the B cells in culture. After this the cells were transferred to a 96-well plate and prepared for transfection.

### Isolation and In Vitro Activation of Human Peripheral Blood B and T Lymphocytes

Healthy donor PBMCs were made available by the Stanford University Medical Center blood bank and isolated by density-gradient centrifugation using the Ficoll-Hypaque technique (Amersham Biosciences). B cells were then extracted using a positive selection method based on CD19 specific magnetic microbeads (Miltenyi Biotec). T Cells were extracted using a negative selection method based on anti-CD43 magnetic microbeads (Miltenyi Biotec). All specimens were obtained with informed consent in accordance with the Declaration of Helsinki, and this study was approved by Stanford University's Administrative Panels on Human Subjects in Medical Research. Samples were collected from patients by peripheral blood leukapheresis and were cryopreserved. After isolation, T cells were resuspended in AIM V serum-free medium (ThermoFisher) supplemented with recombinant human IL-2 at 3000 U/ml (ThermoFisher), anti-CD3 mAb at 1 μg/mL (BD Pharmingen, mouse anti-human NA/LE), anti-CD28 mAb at 5 μg/mL (BD Pharmingen, mouse anti-human NA/LE) and 2-mercaptoethanol (55 uM, Gibco). Once isolated, B cells were resuspended in 10% FCS RPMI supplemented with recombinant human IL-4 at 20 ng/m (Invivogen) and anti-CD40 mAb at 1 μg/ml (BD Pharmingen, mouse anti-human NA/LE). Both B and T cells were cultured in their respective activation media for 24 hours at 37° C. in a 5% CO2 atmosphere, after which the cells were transferred to a 96 well plate and prepared for transfection.

### Flow Cytometry

The following fluorochrome-conjugated rat anti-mouse mAbs were used for flow cytometry: B220-PerCP-Cy5.5, CD21/CD35-FITC, CD11b-BV605 IgD-BV786, CD3-BV786, CD49b-eFlour450, CD3-PE, S1PR1-PE, CD8-PerCP-Cy5,5, CD4-FITC, CD3-APC, CD19-PE, CD11b-PerCPCy5.5, CD69-PE, and rat isotype controls for the listed fluorochromes and antibodies. The following fluorochrome-conjugated mouse anti-human antibodies were used for flow cytometry: APC-S1PR1, PerCpCy5.5-CD8, PE-CD4 and mouse isotype controls for the listed fluorochromes and antibodies. An anti-CD16/32 rat anti-mouse unconjugated antibody was used to block Fc receptor binding sites and prevent nonspecific staining. All the above antibodies were purchased from either BD Biosciences, Invitrogen, or eBioscience, except for the mouse anti-human APC-S1PR1 and the rat anti-mouse PE-S1PR1 antibodies which were purchased from R&D Systems. All surface marker staining was done for 20 minutes at room temperature. Cells were surface stained in wash buffer [PBS, 0.5% BSA (Sigma), and 0.01% sodium azide], either fixed in 2% paraformaldehyde or run fresh, and analyzed by flow cytometry on a FACSCalibur or LSR II System (BD Biosciences). Data was analyzed using either Cytobank (Cytobank Inc.) or FlowJo version 10.0 (FlowJo).

mRNA

EGFP-mRNA (5 meC, Ψ, L-6101), Fluc mRNA (5meC, Ψ, L-6107) and Cy5-Fluc mRNA (5meC, Ψ, L-7702) were purchased from TriLink BioTechonologies Inc.

### General CART Formulation Methods

Deprotected CARTs were dissolved in dimethyl sulfoxide (DMSO) to prepare 2 or 5 mM solutions for in vitro or in vivo applications, respectively. CARTs were formulated with mRNA at a 10:1 cation: anion charge ratio assuming full protonation of the CART and full deprotonation of the oligonucleotides. Formulations were made by mixing the reagents for 20 seconds in acidic PBS (pH adjusted to 5.5 by addition of 0.1 mol/L HCl) in a total volume of 56-250 μl, followed by a brief spin in a tabletop centrifuge. In all experiments, mRNA was first mixed with PBS (pH 5.5) with subsequent addition of the CART (solution in DMSO). The resulting mRNA/CART formulation was then used immediately for in vitro or in vivo experiments. Table 1 below shows size and Zeta potential measurements. Size (dynamic light scattering, DLS) and zeta potential (phase-analysis light scattering, PALS) measurements were performed using a Brookhaven Instruments NanoBrook Omni. For size measurements the effective diameter is reported. For both types of measurements, first a 2 mM solution of CART was added into a solution containing 0.84 μg FLuc mRNA (to achieve a 10:1+/−charge ratio) in a volume of pH 5.5 PBS to give a final total volume of 20.2 uL. For DLS measurements this mixture was then diluted with 2 mL ultrapure water and transferred to a polystyrene cuvette, data was collected at 90° and analyzed by the CONTIN algorithm. For each DLS sample three measurements of 60 second duration were recorded. For PALS measurements the mixture was diluted in 1.3 mL ultrapure water and transferred to a polystyrene cuvette, data were analyzed with a Smoluchowski model. For each PALS sample five measurements of three cycles each were recorded after 2 min of equilibration. All values are reported as the combined average and error (expressed as +SD) of three separate formulations. Encapsulation efficiency was measured by the fluorescence of the RNA-specific Qubit RNA HS dye (Q32852; Invitrogen). Briefly, mRNA was complexed with CART as described above using 840 ng of RNA and enough CART for a net 10:1 (cation: anion) ratio in a volume of 20 uL. To this was added 0.73 mL of RNasefree deionized water followed by 5 μL of Qubit reagent. The fluorescence of the solution was immediately measuring using an excitation wavelength of 630 (4-nm slit width) and emission wavelength of 680 nm (5-nm slit width). Percent encapsulation was determined by subtracting the fluorescence of the Qubit dye in the presence of CART 2 alone (no mRNA, 100% encapsulation) and normalizing to the fluorescence of Qubit with uncomplexed RNA (no CART, 0% encapsulation). Each of the three CARTs showed encapsulation of 95% or higher. Finally, the effects on cell viability were determined using an MTT assay. Briefly, cellular viability following treatment with CART-mRNA complexes was assayed using a PrestoBlue cell viability assay (Invitrogen). Briefly, HeLa cells were seeded at 5000 cells per well, then treated with CART-RNA complexes at a dose of 10 ng (2 ng/1000 cells). After 4 hours, 10 μL of PrestoBlue reagent (Invitrogen) was added to each well. Cells were incubated for an additional 30 min at 37° C. and then fluorescence was measured (ex. 550 nm, em. 600 nm). Untreated cells were included in the panel as a control (100% viability) and treated cell populations were normalized against that fluorescence intensity. There were no detrimental effects on cell viability (data not shown).

TABLE 1
Size and Zeta potential measurements

| | Effective | | Zeta Potential |
|---|---|---|---|
| CART/mRNA NP | diameter (nm) | PDI | (mV) |
| CART 1/mRNA NPS | 157 ± 14 | 0.142 ± 0.038 | 59 ± 16 |
| CART 2/mRNA NPS | 160 ± 12 | 0.231 ± 0.027 | 80 ± 13 |
| CART 3/mRNA NPS | 146 ± 3 | 0.141 ± 0.018 | 52 ± 16 |
| CART 4/mRNA NPS | 137 ± 9 | 0.201 ± 0.022 | 64 ± 14 |

### CART In Vitro Transfection

For all the in vitro transfection experiments, immediately before treatment the cells were washed twice with PBS, resuspended in serum-free media (RPMI or F-12K in case of CHO cells) and then seeded at a concentration of 1×10\hat{ }5 cells per well in a total volume of 40 uL. Then CART formulations were prepared by adding 2.8 μg of mRNA (2.8 uL of a 1 mg/mL stock) to 49.3 uL of PBS (pH 5.5). To this, 3.92 uL of CART (from a 2 mmol/L stock in DMSO) was added and mixed for 20 seconds obtaining a solution prepared in excess for 14 replicates. Then, 4 uL of the mixed mRNA/CART solution was immediately added to each well of a 96-well plate, obtaining either 6 or 12 replicates per condition and resulting in a final mRNA dose of 200 ng/well. This was incubated for 4 hours at 37° C. (5% CO2) after which the transfection reaction was quenched, and mRNA expression analyzed based on the specific gene reporter assay employed as described below.

### Free FTY720 Pre-Treatment

When exposure to free FTY720 prior to transfection was required, cells were resuspended in 1.4 mL of 10% FCS RPMI media in FACS tubes at a cell density of 1×10\hat{ }6/mL and to this FTY720 at either 5 nM or 5 pM was added. The cells were then directly seeded in a 96-well plate at a density of 1×10\hat{ }5 cells/well in a volume of 40 uL and then incubated at 37° C. (5% CO₂). After 30 minutes of incubation, the plate was centrifuged, the supernatant removed and the pelleted cells resuspended in 40 uL/well of serum-free media, ready for treatment with different mRNA/CART polyplexes as described above.

EGFP mRNA Delivery and Expression into Cultured B and T Lymphocytes

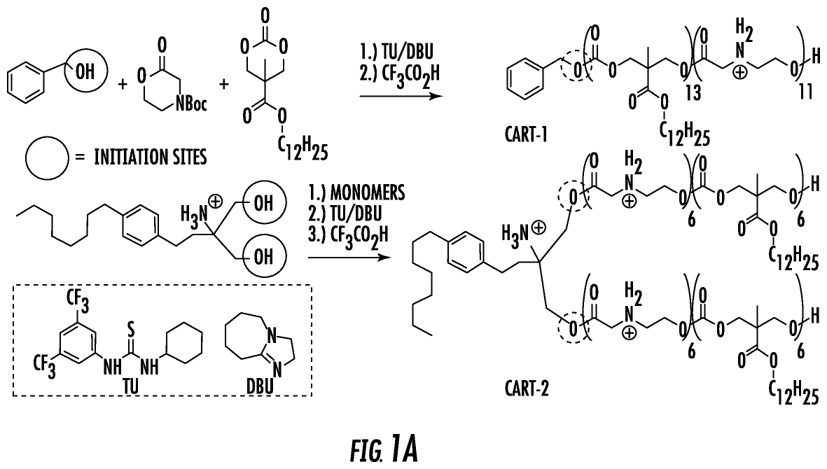

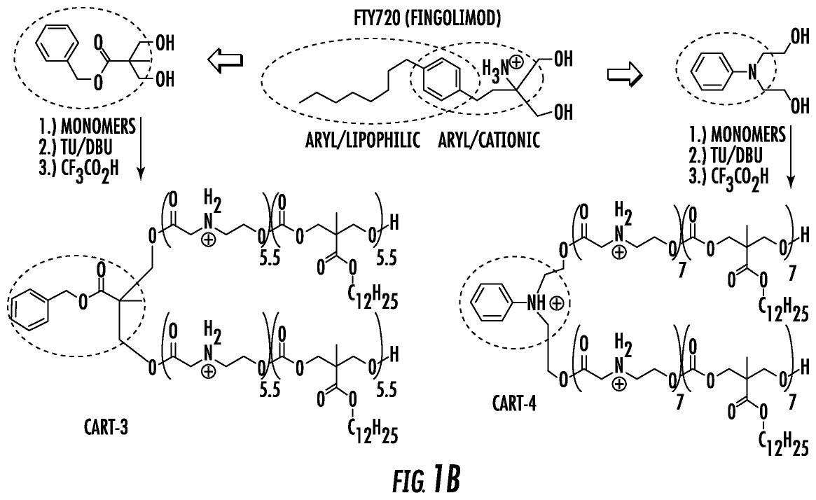

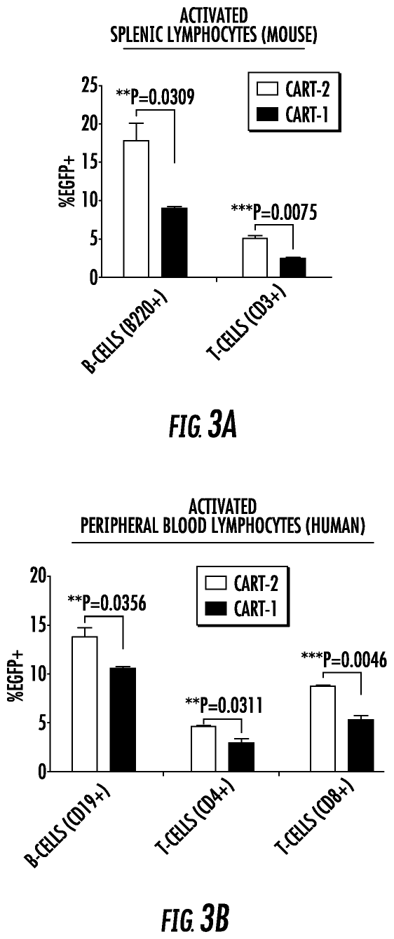

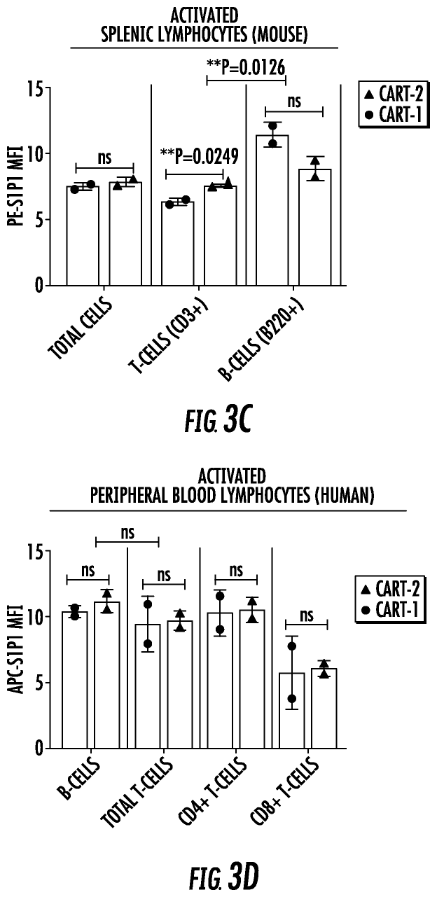

B and T lymphocytes were prepared as above and incubated with the transfection mix for 4 hours at 37° C. (5% CO₂) after which they were resuspended in 10% FCS RPMI media to quench the transfection reaction. At this point, the cells were either immediately analyzed (FIG. 1C) or left in culture for additional 24 hours before analysis (FIG. 3). To measure GFP expression, the plates were centrifuged, the supernatant removed, and the cells resuspended in PBS (150 μL/well). Then, for each experimental condition, cells from each of the 12 replicate wells were collected in 2 FACS tubes that were then analyzed in parallel through flow cytometry to quantify GFP expression (LSR-II system, BD Biosciences). Cells treated with EGFP mRNA only were used as negative controls.

Fluc mRNA Delivery and Expression into Jurkat, CHO and K562 Cells

Jurkat, CHO and K562 cells were prepared as above and incubated with the transfection mix for 4 hours at 37° C. (5% CO2) after which D-Luciferin (Biosynth AG) at a concentration of 1 mM was added directly to the wells. The cells were then incubated at 37° C. for 10 minutes and bioluminescence was measured immediately after through a single-mode microplate luminescence reader (SpectraMax L, Molecular Devices). Cells treated with Fluc mRNA only were used as negative controls. For each condition, bioluminescence was expressed as Average Relative Luminescence Units (RLUs) calculated as:

$$[ Average luminescence for Fluc mRNA - CART treated wells ( n = 6 or 12 ) ] - [ \text{} Average luminescence for Fluc mRNA treated well ( n = 6 or 12 ) ]$$

### Error is Expressed as ±SD.

### CART In Vivo Transfection

For in vivo administration, CART formulations were prepared adding 18.75 μg of mRNA (18.75 uL of a 1 mg/mL stock) to 205 uL of PBS (pH 5.5) and to this 26.22 uL of CART (from a 2 mmol/L stock in DMSO) were added and mixed for 20 seconds. A total of 100 uL of this formulation was administered i.v. into the tail vein of each mouse, injecting two mice at a time per each treatment group and resulting in a final mRNA dose of 7.5 ug/mouse. CART formulations were injected immediately after mixing (within 20 s).

### In Vivo Bioluminescence

Six hours after administration of Fluc mRNA-CART complexes, mice were anesthetized with isoflurane gas and D-Luciferin was injected i.p. at a dose of 150 mg/kg. Fluc expression was measured as whole-body bioluminescence 10 minutes after luciferin injection using an in vivo optical imaging system (IVIS 100; Xenogen Corp.). During image recording, mice were kept under anesthesia with isoflurane delivered via a nose cone, and their body temperature was maintained at 37° C. Image analysis was performed with Living Image Software (PerkinElmer).

### In Vivo Cy5-mRNA Delivery to Murine Splenocytes

Formulations of Cy5-tagged Fluc mRNA and CARTs were prepared and administered i.v. as above. After 2 hours, the mice were sacrificed, the spleen extracted and dissociated to a single cell suspension with a 70 uM cell strainer. Cells were recollected in serum-free RPMI, centrifuged, and resuspended in ACK buffer for 10 minutes to lyse the RBCs. After this, the cells were washed twice in PBS and then divided into three FACS tubes per treatment group. The cells were then stained with different lineage specific antibodies and analyzed through flow cytometry to determine the % of Cy5+ cells for each population.

### Lymphocyte Depletion Assay

Blood was drawn from the submandibular vein of the mice immediately before and 24 hours after treatment with Fluc mRNA-CART complexes prepared and administered as above. In both cases approximately 1 mL of venous blood was collected per mouse into FACS tubes pre-filled with 500 μL of Ca²⁺-EDTA. The blood samples were then centrifuged, the supernatant removed, and the pellet resuspended in ACK lysis buffer for 10 minutes. Then the remaining PBMCs were washed twice with PBS and prepared for flow cytometry analysis.

### Statistical Analysis

The Prism software (GraphPad) was used to determine the statistical significance of differences between groups using the unpaired Student's t-test. P values<0.05 were considered as statistically significant.

### Results and Discussion

Rationale for the synthesis of FTY720-CART

The previously reported benzyl-alcohol initiated CART featuring dodecyl lipid side-chains (CART-1, FIG. 1A) is a robust and effective gene delivery vehicle (DV). Here, we set out to design a CART delivery vehicle that incorporates an FTY720 ligand (fingolimod) inserted into the oligomeric backbone, intended to direct the CART preferentially to S1PR1 overexpressing B cells and T cells and induce significant biological activity of those immune cells consistent with the known biological activity of fingolimod.

We synthesized biodegradable FTY720-containing CARTs by the ring-opening polymerization of lactone and carbonate monomers initiated by an N-boc-protected FTY720 diol, which, because of the two alcohol initiation sites, generates a triblock structure (i.e. short oligomers from both ends of the FTY720 molecule) named FTY720-CART, and from now on referred here as CART-2 (FIG. 1A). The oligomerization was followed by global deprotection with trifluoroacetic acid (TFA) to reveal the fingolimod moiety as well as the ionizable alpha-amino ester groups of the amphiphilic oligomers. Though it is known that singly phosphorylated FTY720 has high affinity for the S1PR1 receptor, we predicted that the structure of FTY720 in the center of the oligomer would be sufficient to direct CART-2 to S1PR1 .

From end-group analysis by 1H NMR of the protected material, the FTY720-initiated CART-2 contains 12 total cationic monomers and 12 lipophilic monomers per initiator (CART-2=FTY720, A12:D12). In these studies, the previously reported deblock CART-1 is included for comparison, which contains 13 lipophilic monomers and 11 cationic monomers per initiator (CART-1=Benzyl, D13: A11) (Mckinlay et al., Proc Natl Acad Sci USA. 2017; 114(4):E448-E456). Additionally, we synthesized triblock analogues of the FTY720-initiated CART-2 to study specific effects of the FTY720 moiety (vide infra). One analog is initiated with benzyl-2,2-bis (hydroxymethyl) propionate, B-DMPA (CART-3, FIG. 1B) and contains 11 cationic monomers and 11 lipophilic monomers per initiator (data from protected material, CART-3=B-DMPA, A11:D11). The other analog, initiated with N-phenyldiethanolamine, PhDEA (CART-4, FIG. 1B) contains 14 cationic monomers and 14 lipophilic monomers per initiator (data from protected material, CART-4=PhDEA, A14:D14).

A. Synthesis of CART-2: organocatalysts TU=1-(3,5bis(trifluoromethyl)phenyl)-3-cyclohexylthiourea, DBU=1,8-Diazabicyclo[5.4.0]undec-7-ene, (CART-1=Benzyl, D13: A11; CART-2=FTY720, A12: D12). DP determined by 1H NMR of the protected material. B. Synthesis of CART-3 and CART-4: Aryl/Lipophilic FTY720 analogue used for synthesis of CART-3: benzyl-2,2 bis(hydroxymethyl) propionate (B-DMPA); Aryl/Cationic FTY720 analogue used for synthesis of CART-4: N-phenyldiethanolamine (PhDEA). (CART-3=B-DMPA, A11: D11; CART-4=PhDEA, A14: D14). DP determined by 1H NMR of the protected material.

### CART-2 Outperforms CART-1 Via an S1PR1-Mediated Mechanism

We have previously demonstrated that CARTs effectively encapsulate and deliver mRNA to the cellular cytoplasm, both in vitro and in vivo, via a mechanism dependent on endocytosis and subsequent escape from the endosome (Mckinlay et al., Proc Natl Acad Sci USA. 2017; 114 (4): E448-E456). We also showed that changes in the chemical structure of the CARTs can alter the organs and cells preferentially targeted by the mRNA/CART polyplexes (Mckinlay et al., Proc Natl Acad Sci USA. 2018; 115 (26): E5859-E5866. The chemical versatility of CARTs allows us to use a large array of small molecules as initiators in their synthesis. An advantage of functionalizing CARTs with a small molecule ligand is to selectively direct the mRNA/CART polyplexes to cells that express the receptor for the small molecule ligand. This ligand-receptor interaction should in theory preferentially increase mRNA delivery to and expression of the functionalized CART in cells that harbor a surface receptor for the small molecule ligand. To test this theory, we synthetized an FTY720-conjugated CART, referred to herein as “CART-2”. FTY720 is a Sphingosine 1-Phosphate (SIP) analogue and acts as a superagonist of the S1PR1 receptor, which in vivo is highly expressed on T and B lymphocytes in secondary lymphoid organs.

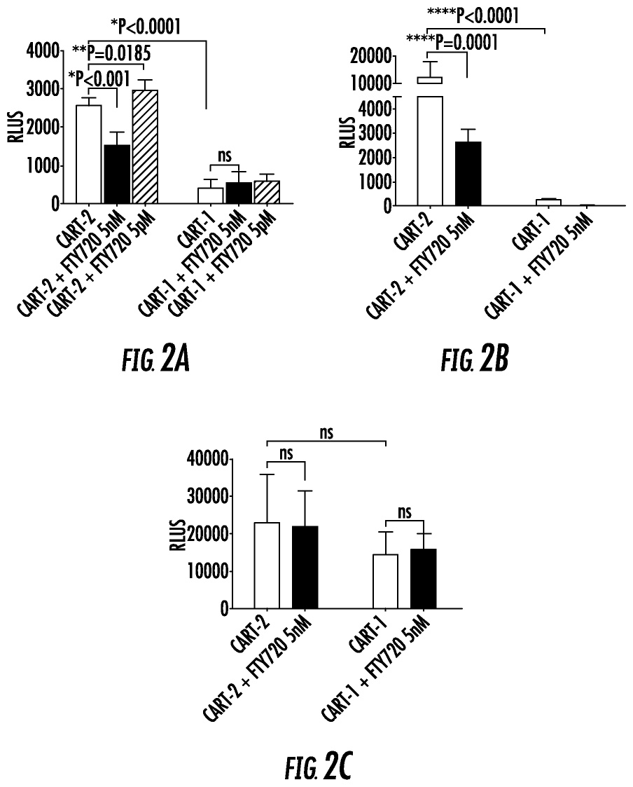

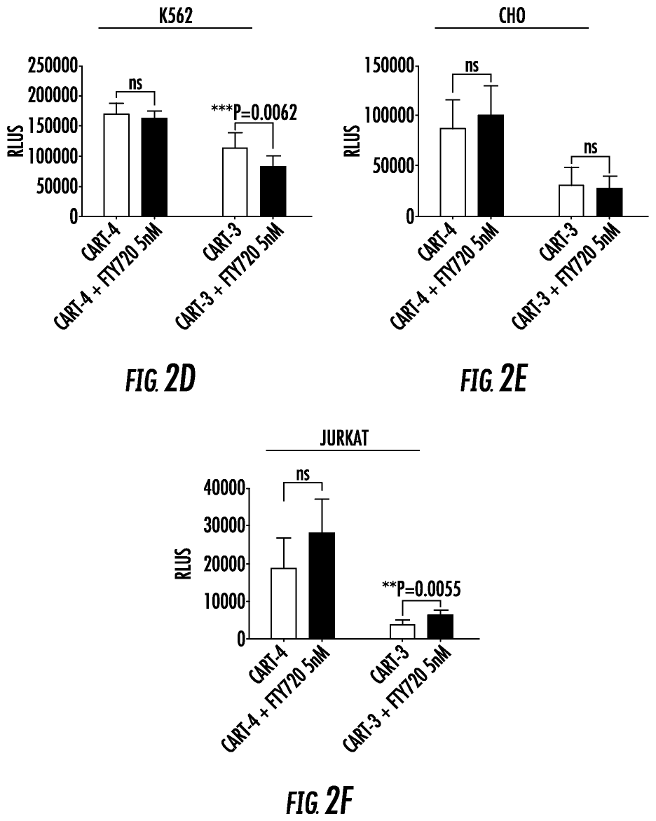

To test if the novel CART-2 molecule more efficiently transfects cells that express S1PR1, we complexed mRNA coding for the reporter gene firefly luciferase (Fluc) to either CART-2 or its non-FTY720-conjugated analogue, CART-1. We then treated immortalized cells that either express or do not express S1PR1 with the two different Fluc mRNA/CART complexes (FIG. 2). Specifically, we used the Jurkat and K562 cell lines as an S1PR1 expressing cell model (S1PR1+), and the CHO cell line as a model that naturally lacks expression for S1PR1 (S1PR1−). We then measured mRNA expression in cells treated with CART/mRNA complexes as relative bioluminescence compared to cells treated with naked Fluc mRNA, used as negative controls. In Jurkat and K562 cells, the CART-2 achieved a 6-fold and a 48-fold increase in Fluc expression respectively, compared to CART-1 (FIG. 2A-B). In contrast, in CHO cells there was no difference in bioluminescence between cells treated with CART-1 and those treated with CART-2 (FIG. 2C). We also tested CART-2 using an Enhanced Green Fluorescent Protein (EGFP) reporter readout in primary B lymphocytes isolated from the spleen of BALB/c mice. Splenic murine B lymphocytes are known to express S1PR1 in vivo, and we confirmed that the primary cells retained expression of S1PR1 ex vivo. Animals were sacrificed and the spleen extracted and rendered into a single cell suspension. Cells were then rested for 12 hours before transfection with EGFP mRNA complexed to either CART-2 or CART-1. After 4 hours, the cells were stained for surface lineage markers and analyzed by flow cytometry to quantify EGFP expression in B cells. Interestingly, EGFP expression was 30% higher in cells treated with CART-2 compared to CART-1 even though statistical significance was not reached (P=0.0543, data not shown).

Then, to determine if the superiority of CART-2 in S1PR1 expressing cells is mediated by a specific interaction with the S1PR1 receptor, we exposed cells to free FTY720 for 30 minutes before treatment with different mRNA/CART complexes. After exposure to free FTY720 at 5 nM (EC50), there was a 40% reduction in Fluc expression in Jurkat cells treated with CART-2 but no change in luminescence in those treated with CART-1 complexes (FIG. 2A). Also, when Jurkat cells were exposed to free FTY720 at 5 pM, which is a dose 10-fold lower than the EC50 dose, for 30 minutes, there was no reduction in bioluminescence after treatment with CART-2, suggesting a dose-dependent phenomenon. In K562 cells we observed a reduction in Fluc expression after exposure to FTY720 both in those treated with CART-2 and those treated with CART-1 (FIG. 2B). Instead, in the S1PR1 negative CHO cells, pre-exposure to free FTY720 at the EC50 does of 5 nM did not affect Fluc expression after treatment with either CART-2 or CART-1 (FIG. 2C). Similar results were observed in primary splenic murine B lymphocytes, where a 30 minutes pre-exposure to free FTY720 at the EC50 does of 5 nM caused a 1.9-fold reduction in EGFP expression among cells treated with CART-2 (data not shown). In contrast, in B cells treated with CART-1 we observed a 32% increase in GFP expression with pre-exposure to free FTY720.

One explanation for these results could be the previously demonstrated FTY720-induced internalization of SIPR1. To test if this was the case in our experiments, we again exposed CHO, K562 and Jurkat cells, as well as primary splenic murine B lymphocytes, to FTY720 at 5 nM for 30 minutes. Immediately after, the cells were collected and stained with an S1PR1-APC antibody for flow cytometry quantification of surface S1PR1 expression. Notably, we observed that surface expression of S1PR1 is not reduced after exposure to FTY720 in any of the cellular models we employed. Another possible mechanism is that free FTY720 saturates the binding sites on S1PR1 preventing its interaction with the mRNA/CART-2 complex.

Using FTY720 as the initiator in the synthesis of CARTs can potentially change the tertiary structure of the CART-mRNA particles. It could thus be argued that a change in the tertiary structure of the mRNA/CART complexes, rather than a specific ligand-receptor interaction, is responsible for the increased transfection observed with CART-2 in S1PR1 compared to S1PR1-negative cells. To address this issue, as described above, we synthesized CART-3 and CART-4 as structural analogs of CART-2. Both CART-3 and CART-4 exhibit a triblock structure analogous to CART-2 but feature initiator moieties only partially resembling FTY720. Again, we treated Jurkats, K562 and CHO cells in vitro with Fluc mRNA complexed to either CART-3 or CART-4, with and without pre-exposure to free FTY720 at 5 nM for 30 minutes. CART-3 and CART-4 were effective in all three cell lines, showing variable degrees of Fluc expression, but in contrast to the behavior observed for CART-2, luciferase expression did not change in either K562 or CHO cells upon pre-exposure to free FTY720 (FIG. 2D, 2E). For Jurkat cells we observed a measurable increase (˜50%) in Fluc expression after pre-exposure to free FTY720, in both cells treated with CART-3 and CART-4 (FIG. 2F). Hence, pre-exposure to free FTY720 impairs the transfection efficiency of CART-2 but not of CART-3 and CART-4, and this is true only in S1PR1-expressing cells

These results suggest an S1PR1-specific interaction responsible for the superior mRNA delivery and translation observed in S1PR1 expressing cells with mRNA/CART-2 compared to mRNA/CART-1 complexes. These findings are significant since, to our knowledge, CART-2 would be the first mRNA delivery vehicle to exploit the specific interaction of a small molecule drug with its coupled receptor to release the cargo mRNA more efficiently and specifically into its target cells. The concept of using small molecules to target cell populations of interest has been used with peptide-targeting ligands and sugar-based compounds, such as GalNac and galactose. However, incorporating a single, receptor specific, small molecule drug into oligomeric DVs or LNPs has not been previously reported. Overall, our results offer proof-of-concept that receptor-mediated mRNA delivery could be a feasible and effective approach to direct mRNA to a specific cell type.

CART-2 Transfects Primary Human and Murine Lymphocytes More Efficiently than CART-1

B and T lymphocytes use the S1PR1 receptor to sense the gradient of SIP that is naturally more concentrated in the blood compared to secondary lymphoid organs. S1P acts as a chemoattractant for B and T cells mediating their egress from the spleen and lymph nodes (LNs) to the bloodstream. Also, S1PR1 is usually upregulated after activation of lymphocytes to favor their exit from secondary lymphoid organs and accumulation to areas of active immune response. Given the key role that S1PR1 plays in the biology of B and T cells, we asked if CART-2 could more efficiently transfect primary lymphocytes compared to its non-FTY720-initiated analogue CART-1. To address this question, we used both murine and human primary B and T lymphocytes.

First, we extracted primary B and T lymphocytes from a murine spleen and co-cultured them in vitro for 24 hours with anti-CD3 and anti-CD28 antibodies added to the growth medium to obtain direct activation of T cells and bystander activation of B cells. Cells were then treated with EGFP mRNA complexed to either CART-1 or CART-2 (FIG. 3A). The transfection reaction was quenched 4 hours later, and the cells were then cultured for additional 24 hours to allow full expression of EGFP, after which they were analyzed through flow cytometry to determine EGFP expression in both T cells (CD3+) and B cells (B220+). In this case, we observed a 2-fold increase in EGFP expression in both B and T lymphocytes after treatment with CART-2 compared to CART-1. Cell activation was confirmed by measuring CD69 expression on both B and T cells. Comparable results were observed in primary human lymphocytes. In this case peripheral blood mononuclear cells (PBMCs) from healthy donors were thawed and B and T lymphocytes isolated with lineage-specific magnetic microbeads. After isolation, B cells were cultured for 12 hours in presence of human IL-4 (20 ng/mL) and anti-CD40 (1 μg/mL) to obtain direct B cell activation. Similarly, after isolation, T cells were cultured for 12 hours and activated by the addition to the culture medium of anti-CD3 (1 μg/mL), anti-CD28 (5 μg/mL) and IL-2 (3000 UI/mL). T and B lymphocytes were then treated in parallel with EGFP mRNA complexed to either CART-2 or CART-1 and the transfection reaction was quenched 4 hours later. The cells were cultured for an additional 24 hours and EGFP expression was measured through flow cytometry (FIG. 3B). In activated human B lymphocytes EGFP expression was 38% higher in those treated with CART-2 compared to CART-1. Also, in activated human T cells treatment with CART-2 resulted in a 55% and 64% increase in EGFP expression respectively in the CD4+ and CD8+ sub-populations, compared to CART-1.

The expression of S1PR1 was confirmed through flow cytometry on total activated murine splenic lymphocytes (FIG. 3C) with higher levels on B cells compared to T cells, similar levels on B-cells treated with CART-1 compared to CART-2 and higher levels on T cells treated with CART-2 compared to CART-1. We also confirmed S1PR1 expression on activated human B-cells (FIG. 3D) and activated human T-cells (FIG. 3E), with no difference in S1PR1 levels between the two populations, as well as between cells treated with CART-2 or CART-1 in either B cells, total T cells, CD4+ T cells or CD8+ T cells.

Overall, these results show that CART-2 can more efficiently transfect primary B and T lymphocytes in vitro compared to its non-FTY720-initiated analogue CART-1. This is particularly significant from a therapeutic standpoint since there is a growing demand for better technologies to transfect lymphocytes in vitro as it is needed, for example, in the development of adoptive T cell cancer immunotherapy strategies.

CART-2 Efficiently Delivers mRNA to Splenic Marginal Zone B Cells and NK Cells In Vivo

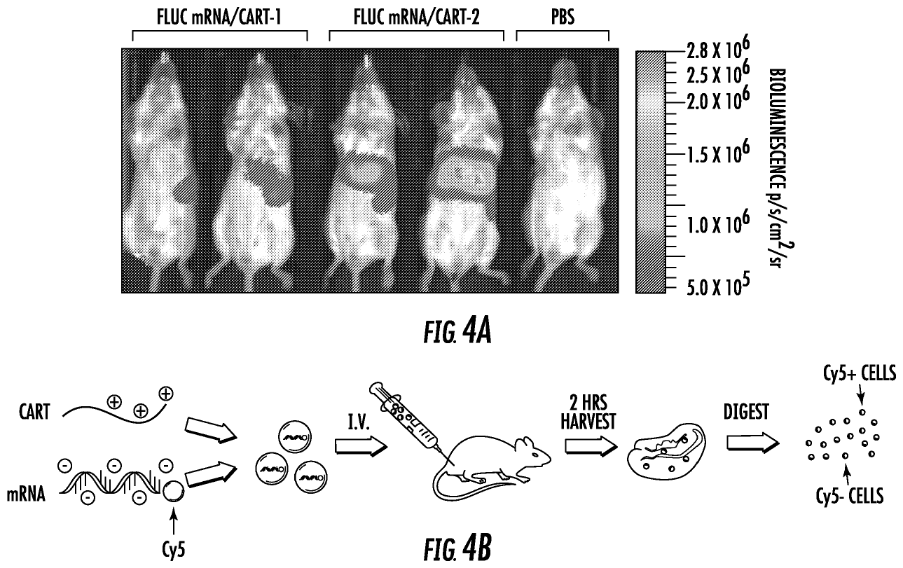

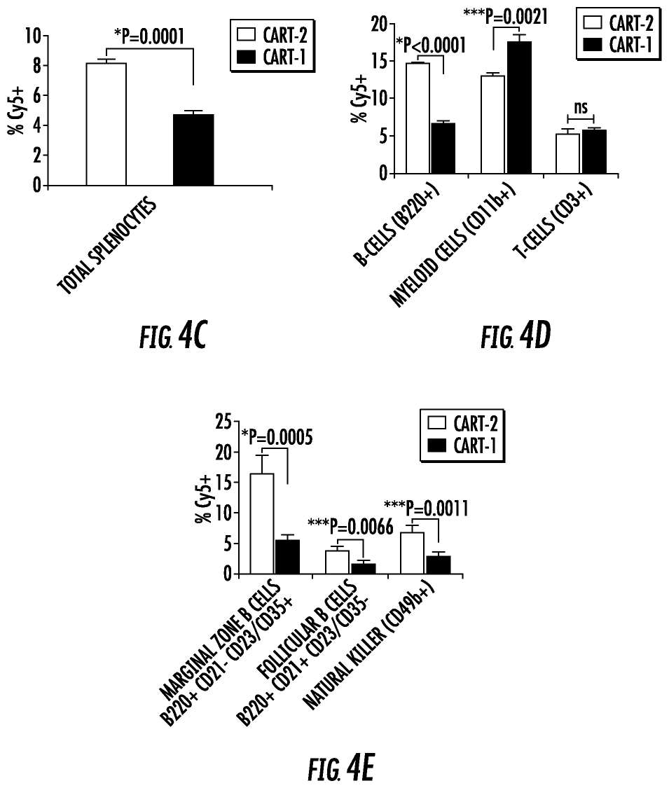

As previously shown, altering the chemistry of CARTs can change the organs and cells targeted by the mRNA/CART complexes in vivo. Thus, we asked if the presence of FTY720 in the structure of the CART would change the organs and cells targeted in vivo by CART-2 compared to its non-FTY720 analogue CART-1. To answer this question, we complexed mRNA coding for Fluc to either CART-2 or CART-1. We then injected the resulting mRNA/CART complexes intravenously (i.v.) to BALB/c mice (FIG. 4A). Expression of the cargo mRNA was measured as whole body bioluminesce (BLI) 6 hours after the injections. The main site of mRNA expression in animals treated with CART-1 was the spleen while the organs transfected in mice treated with CART-2 were mainly the liver, and to a lesser extent the spleen.

Given the key role that the spleen plays in regulating systemic immune responses, we asked if there was a difference in the cells targeted within the spleen by the mRNA/CART-2 compared to the mRNA/CART-1 complexes. To address this question, we used Fluc mRNA conjugated to the Cyanine 5 fluorophore (Cy5-mRNA). The Cy5-mRNA was mixed with either CART-2 or CART-1 and the resulting mixtures were administered i.v. to BALB/c mice (FIG. 4B). The animals were sacrificed 2 hours later, and the spleens extracted to obtain a single cell suspension that was then analyzed through flow cytometry to determine Cy5-mRNA delivery to specific cellular subpopulations. First, we observed that the total percentage of Cy5+ cells in the spleen was 1.8 times higher in animals treated with CART-2 compared to CART-1 (FIG. 4C). We then measured Cy5-mRNA uptake in splenic B cells (CD19+), T cells (CD3+) and myeloid cells (CD11b+) separately (FIG. 4D). In this case, we found no difference in mRNA delivery to T cells between mice treated with CART-2 and those treated with CART-1, while there was a 2.2-fold increase in mRNA delivery to B cells in animals treated with ART-2 compared CART-1, and a 1.3-fold increase in delivery of mRNA to myeloid cells in mice that received CART-1 compared to CART-2.

In the murine spleen, and similarly in the human spleen, there are two main B cell populations, the marginal zone B cells (MZ B) and the follicular B cells (FO B) that differ significantly in terms of S1PR1 expression and responsiveness to SIP in vivo. Specifically, MZ B cells are specialized antigen presenting cells (APCs) that constantly collect and transport antigens from the bloodstream to the follicles where these are processed and presented to FO B cells to elicit a humoral immune response. During this perpetual shuttling between the follicles and the marginal zone, the MZ B cells follow the gradient of SIP through the S1PR1 and SIP3 receptors to migrate from the follicle back to the marginal zone. FO B cells instead show lower expression of S1PR1 and lower responsiveness to SIP compared to the MZ B cells. Given the higher S1PR1 expression in splenic MZ B compared to FO B cells, we asked if there was a difference in the proportion of MZ B cells and FO B cells targeted by CART-2 and CART-1. To answer this question, in a separate experiment we again formulated Cy5-mRNA to either CART-2 or CART-1 and administered the complexes i.v. to BALB/c mice (FIG. 4E). Animals were sacrificed 2 hours later to analyze mRNA delivery to splenic MZ B cells and FO B cells through flow cytometry. MZ B cells were defined as B220+CD23−CD21/CD35+ cells while FO-B cells were defined as B220+ CD23+ CD21/CD35−. In animals treated with CART-2 the average percentage of Cy5+ MZ B cells and Cy5+ FO B cells were respectively 2.9-fold and 2.4-fold higher compared to animals treated with CART-1. Also, the percentage of Cy5+ MZ B cells was 4.9 times higher than that of FO B cells in animals treated with CART-2, while in animals treated with CART-1 the percentage of Cy-5+ MZ B cells was only 3.6 times higher than what seen in FO B cells. In the same experiment, we also looked at mRNA delivery to splenic Natural Killer (NK) cells (CD49b+) that are known to express the SIP5 receptor to which FTY720 has a lower but still significant affinity. In this case we found a 2.4-fold increase in the percentage of splenic Cy5+CD49b+ cells after treatment with CART-2 compared to CART-1.

Overall, these results suggest that conjugating FTY720 to the CART structure can increase mRNA delivery to the spleen of living animals after i.v. administration of the mRNA/CART complexes. Also, within the spleen, CART-2 more efficiently delivered mRNA to splenic MZ B cells and NK cells compared to CART-1, which could be particularly important from a therapeutic standpoint. Indeed, MZ B cells play a significant role as APCs in starting both humoral and cellular immune responses against bloodborne antigens. This subset of B lymphocytes is also involved in the generation of immune tolerance, and dysregulation of MZ B cells has been implicated in the pathophysiology of autoimmune diseases like Systemic Lupus Erythematosus. There is growing evidence that MZ B cells could be targeted for treatment of cancer and infectious diseases. For example, a previous study has shown that it is possible to elicit a systemic antitumor T cellular response in mice after i.v. administration of nanoparticles that selectively deliver tumor-associated antigens to splenic MZ B cells. As far as NK cells, these are a poorly studied and difficult to transfect class of lymphocytes that play a significant role in cancer immune surveillance and offer a new target for cancer immunotherapy. For example, we have recently shown that CARTs can efficiently transfect primary human NK cells in vitro to generate NK-Chimeric Antigen Receptor cells. CART-2 was able to effectively deliver mRNA to 7% of the splenic NK cell population after i.v. administration in mice. This property of CART-2 could be exploited to manipulate NK cells in vivo to generate potent systemic anti-tumor immune responses.

### CART-2 Induces Peripheral T Cell Depletion Similarly to Free FTY720

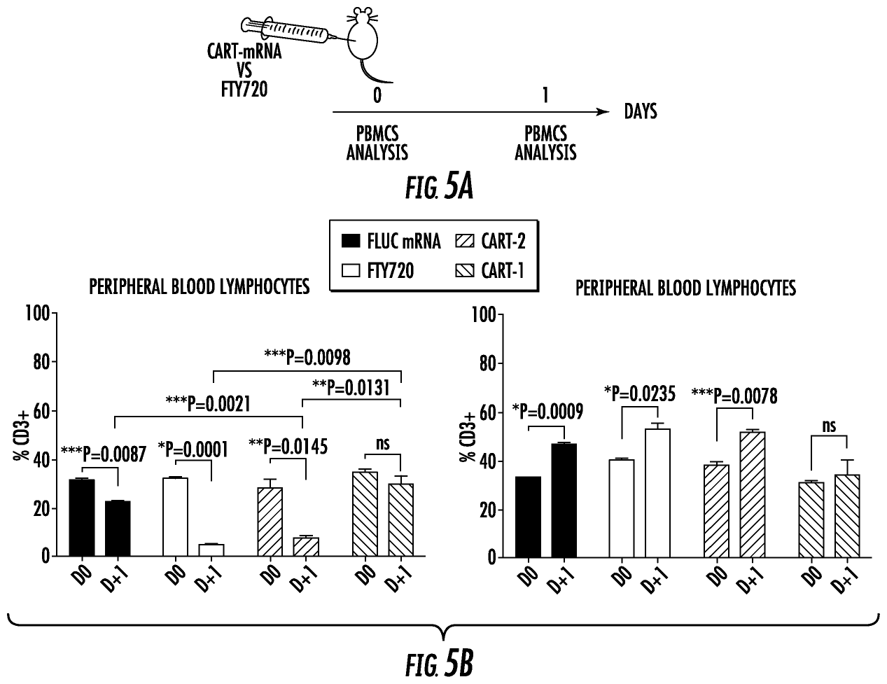

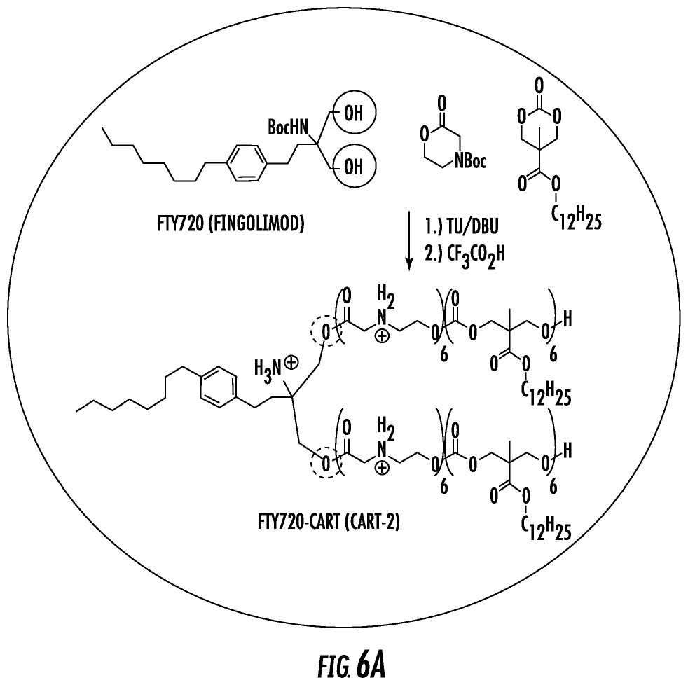

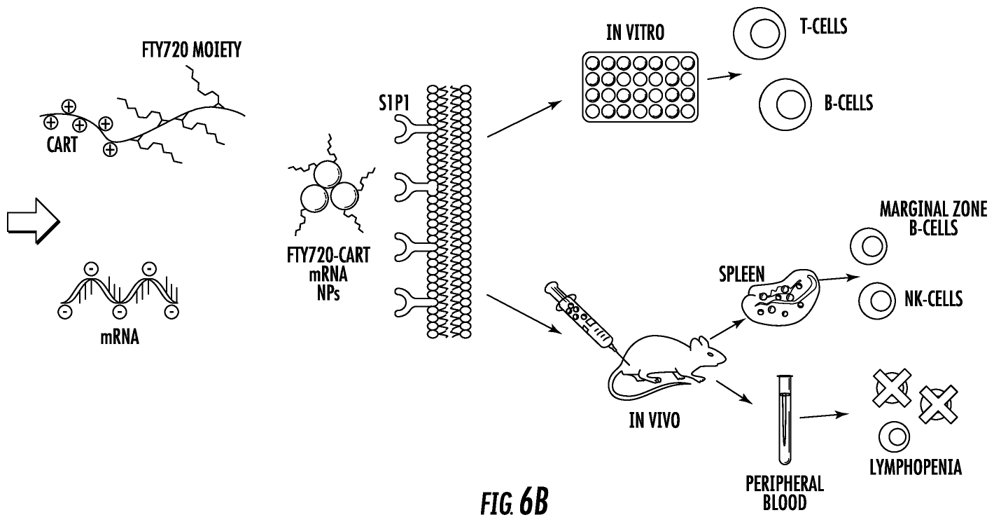

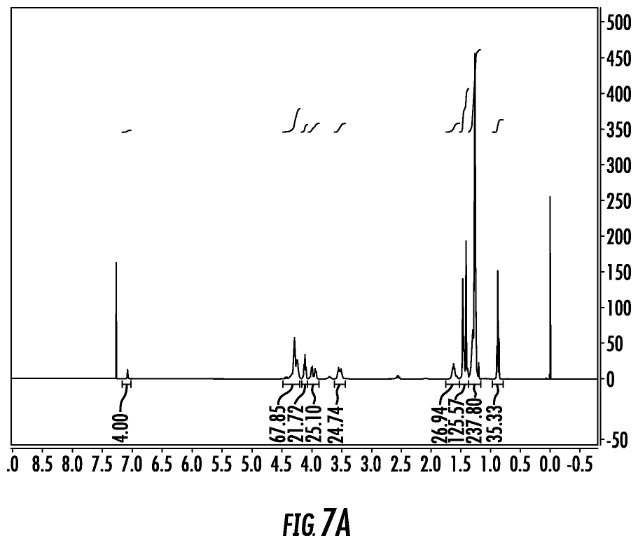

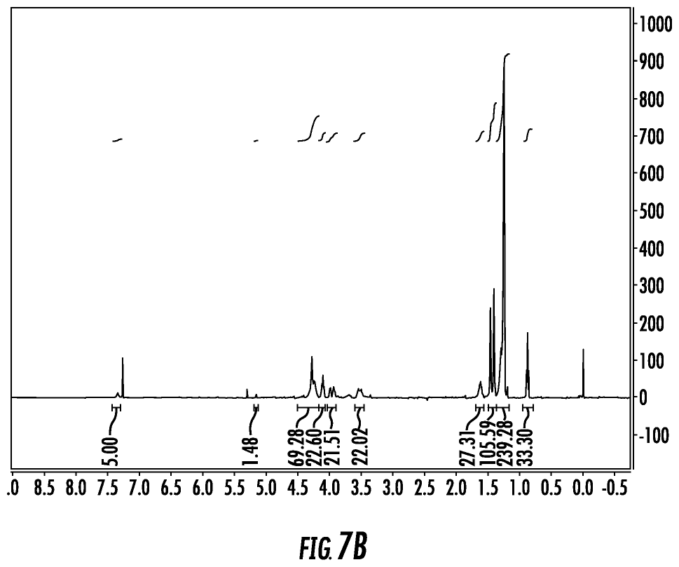

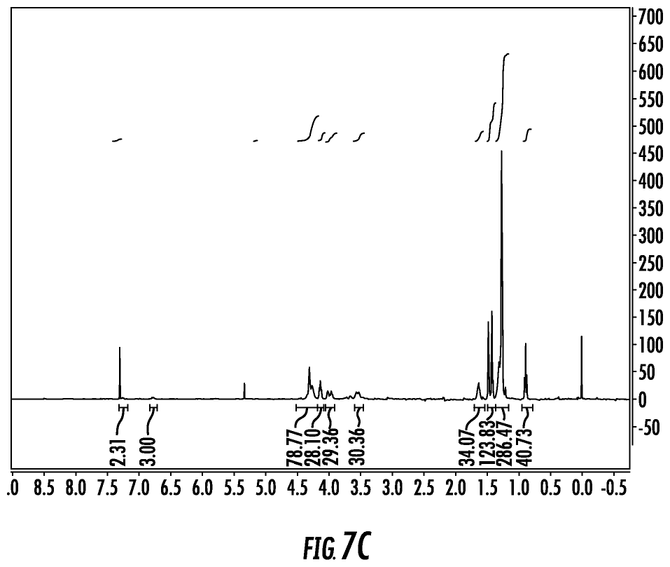

One of the potential benefits of functionalizing an mRNA delivery vehicle with a small molecule is to exploit the biological properties of the small molecule for therapeutic purposes. FTY720 exerts its immunosuppressive effects by segregating T and B cells in secondary lymphoid organs thus interfering with their migration to sites of active immune response. We thus asked if FTY720 can induce lymphopenia in treated animals when present in the structure of CART-2. To answer this question, we treated BALB/c mice with either free FTY720, naked Fluc mRNA or Fluc mRNA complexed to CART-1 or CART-2, all administered i.v. The mice that received free FTY720 were treated with 12.9 μg of FTY720 to match the amount of molecular FTY720 present in each dose of Fluc mRNA/CART-2. Also, the mice that received naked Fluc mRNA were administered the same amount of mRNA present in each mRNA/CART complex (7.5 μg/mouse). Before treatment, mice were bled to measure the baseline levels of circulating T cells (CD3+) and B cells (B220+) calculated through flow cytometry as a percentage of the total peripheral blood lymphocytes. The mice were then bled 24 hours after treatment and the levels of peripheral T and B lymphocytes were measured again (FIG. 5). As expected, in mice treated with free FTY720 we observed an 84% reduction in the percentage of circulating T cells one day after treatment. Similar results were observed in mice treated with Fluc mRNA/CART-2 where there was a 72% reduction in circulating T cells. There was instead no reduction in the proportion of circulating T lymphocytes in animals that received Fluc mRNA/CART-1, while in mice treated with only Fluc mRNA we observed a 27% decrease in the percentage of circulating T cells. This small decrease in the proportion of circulating lymphocytes observed with naked mRNA administration could be an artefact caused by the innate activating properties of naked mRNA. Looking at peripheral B cells, in animals treated with either free FTY720, naked Fluc mRNA or Fluc mRNA/CART-2 we observed an increase in the percentage of circulating B cells 24 hours after treatment, possibly secondary to the concomitant reduction in circulating T cells. In contrast, in animals that received Fluc mRNA/CART-1 there was no change in the percentage of circulating B-cells after treatment.

Hence, animals treated with CART-2 showed almost complete peripheral T cells depletion similar to those that received free FTY720. However, neither free FTY720 nor CART-2 caused a depletion of circulating B cells. One explanation for the lack of effect on circulating B cells is that the dose of FTY720 used in our experiments was 12.9 μg/mouse (approximately 0.7 mg/kg) while previous data show B cell depletion at a FTY720 dose of 2.5 mg/kg per mouse. Also, in prior studies, B cell depletion was observed with daily oral administration to mice over a period of 21 days while in our experiments we administered a single dose of FTY720 intravenously and assessed B cell depletion 24 hours later. Overall, these results suggest that the FTY720 moiety retains, at least in part, its biological activity when present in the structure of CART-2. This is particularly important if we consider the therapeutic role of FTY720 in Multiple Sclerosis. CART-2 could indeed be used to develop mRNA-based therapies, not only for Multiple Sclerosis but for autoimmune disorders in general. For example, one approach could be to deliver mRNA coding for immunosuppressive cytokines to autoreactive T and B cells while at the same time causing lymphopenia through the FTY720 moiety, with a resulting synergistic immunosuppressive effect.

In summary, we describe here utilization of the versatile chemistry of the CART delivery platform to conjugate a single small molecule drug, FTY720, to the core backbone of the CART to generate a triblock co-oligomer. We specifically chose to use FTY720 given its known ability to bind with high affinity to the S1PR1 receptor on T and B lymphocytes in secondary lymphoid organs. We further leveraged the CART platform by generating CARTs initiated with FTY720-mimics (CART-3 and CART-4) to better elucidate the specific effects of the FTY720-initiated CART (CART-2). Our main goal was to create an mRNA DV that was able to deliver mRNA more efficiently and more selectively to lymphocytes for therapeutic purposes, both in vitro and in vivo.

Here we showed that the novel CART-2 is superior to its non FTY720-initiated analogue CART-1 in transfecting cells that express the FTY720 receptor S1PR1 while the two CARTs were equivalent in cells that lack expression of S1PR1. CART-2 was also particularly effective in transfecting activated human and murine B and T cells in vitro which can be significant from a clinical and therapeutic standpoint. Also, transfection with CART-2 was impaired by pre-exposure of target cells to free FTY720, overall suggesting an S1PR1-mediated mechanism for mRNA delivery with CART-2.

As far as the in vivo properties of CART-2, we noticed increased delivery and expression of mRNA to the spleen of mice after intravenous administration and within the spleen, more efficient mRNA delivery to B cells, particularly MZ B cells, and NK cells. These properties of CART-2 are particularly appealing for potential applications in cancer immunotherapy as well as vaccine development and treatment of autoimmune diseases.

Lastly, we found that CART-2 causes depletion of T lymphocytes from the peripheral blood similarly to free FTY720 suggesting that the FTY720 moiety might partially retain its biological activity in the CART structure. This finding further increases the therapeutic potential of the CARTs, with the possibility of achieving synergistic effects between the mRNA cargo and the small molecule conjugated to the CART polymer.

While prior studies have shown that it is possible to increase the efficiency and specificity of mRNA delivery by conjugating receptor-binding peptides or sugar-based compounds to lipid nanoparticles, CART-2 represents to our knowledge the first mRNA delivery vehicle that incorporates a small molecule drug in its structure. Overall, these results encourage using different small molecules as initiators in the synthesis of CARTs to create new mRNA delivery vehicles that can specifically and more potently transfect cells both in vitro and vivo for therapeutic applications in cancer, autoimmune and infectious diseases.

1. A copolymer of Formula (I) comprising a ligand moiety, A, one or two lipophilic polymer blocks, LP¹, LP², and a poly (alpha aminoester) block, IM: 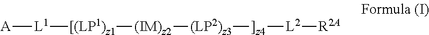
wherein:
A is a ligand moiety that binds to a cell surface receptor;
R^{2A} is hydrogen, OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹ and L² are optional and each L¹ and L² is independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
LP¹ and LP² are each independently 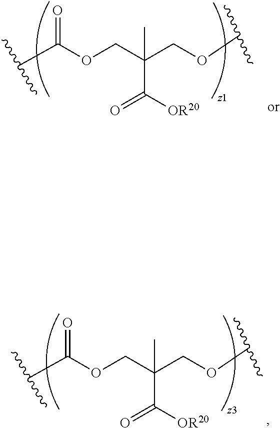
wherein
z1 and z3 are independently from 1 to 100,
wherein at least one of z1 or z3 is not 0; and
R²⁰ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
IM is 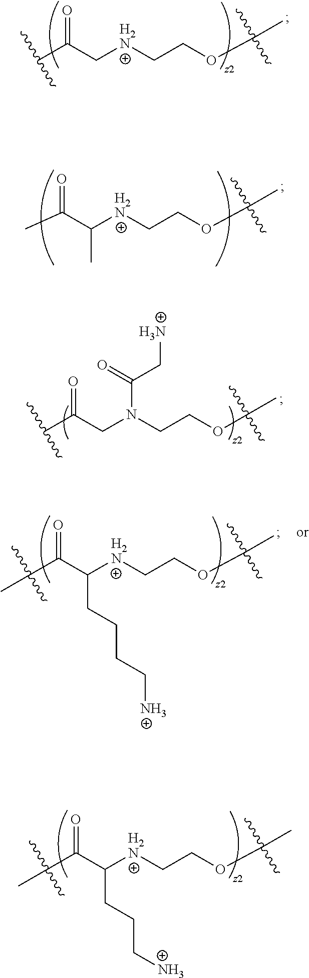
wherein z2 is from 2-100; and
z4 is 1 to 100.

2. The copolymer of claim 1 where A is saccharide, a disaccharide, an oligosaccharide, a liposaccharide, a lipid, a peptide, an antibody, or a small molecule.

3. The copolymer of claim 1 where A is:

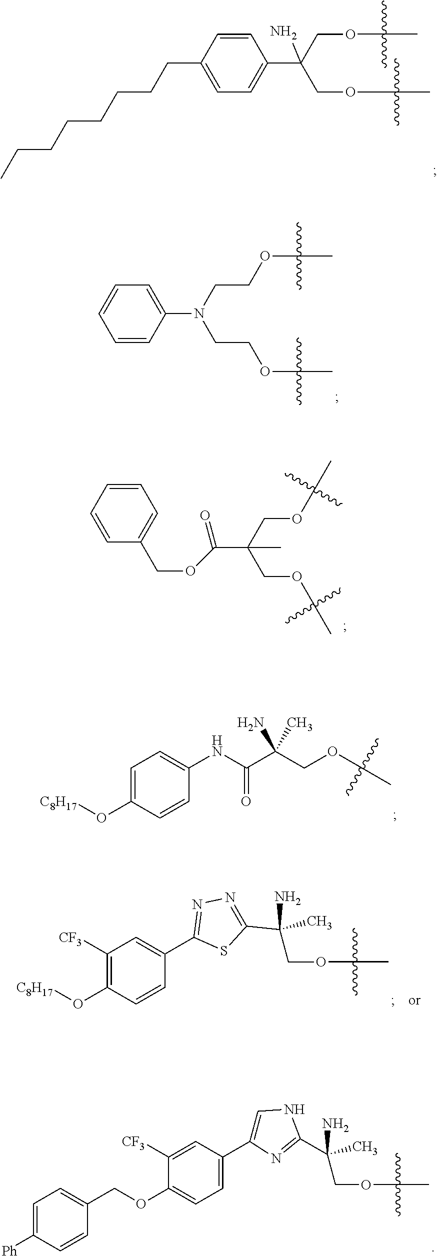

4. The copolymer of claim 1, where A is glucose (beta-D-glucopyranoside) or galactose (alpha-D-galactopyranose).

5. The copolymer of claim 1, where L₁ is:

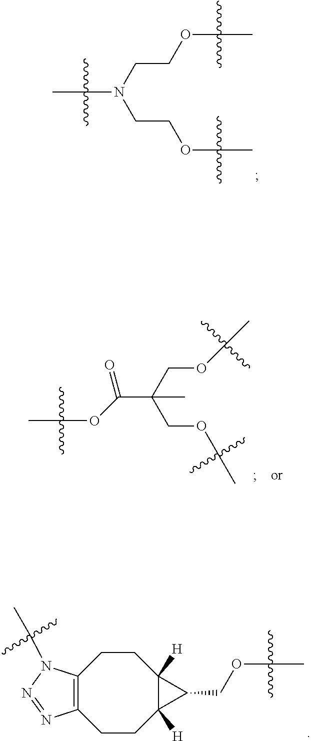

6. The copolymer of claim 1, where
z1 is zero;
z4 is 1;
IM is 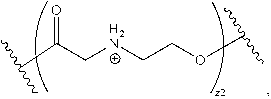
where z2 is from 1 to 100;
and
LP₂ has the formula: 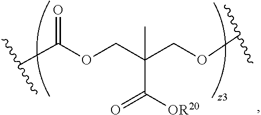
where z3 is from 1 to 100; and
R²⁰ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

7. The copolymer of claim 6, where z2 is 12.

8. The copolymer of claim 6, where R²⁰ is independently hydrogen, unbranched C₁-C₃₀ alkyl, which may be fully saturated, mono- or polyunsaturated, or cholesterol.

9. The copolymer of claim 8, where R²⁰ is independently hydrogen, stearyl, oleyl, linoleyl, dodecyl, nonenyl, or cholesterol.

10. The copolymer of claim 1, where the copolymer has the structure: 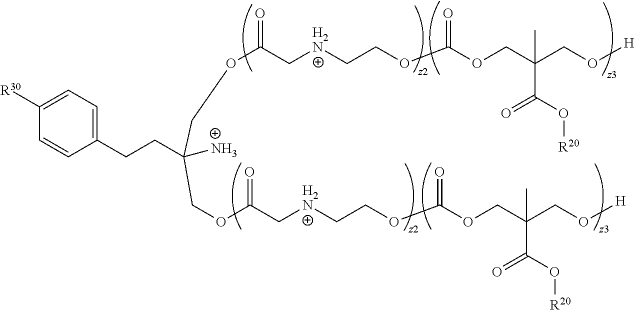
wherein
R³⁰ is unsubstituted C8 alkyl and
R²⁰ is independently hydrogen, unbranched C₁-C₃₀ alkyl, which may be fully saturated, mono- or polyunsaturated, or cholesterol, optionally wherein R²⁰ is independently hydrogen, stearyl, oleyl, linoleyl, dodecyl, nonenyl, or cholesterol.

11. The copolymer of claim 1 having a structure of Formula (Ia):

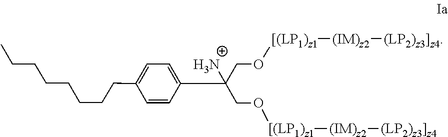

12. The copolymer of claim 11 having the structure of

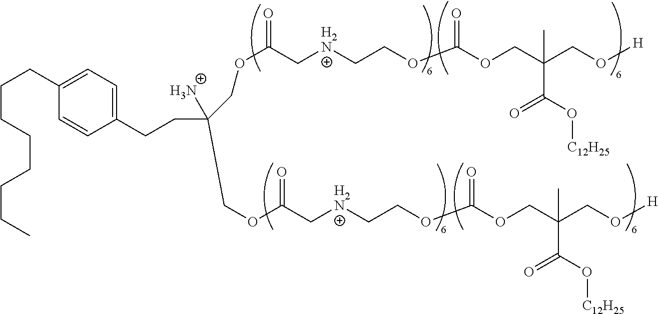

13. The copolymer of claim 1, having the structure of

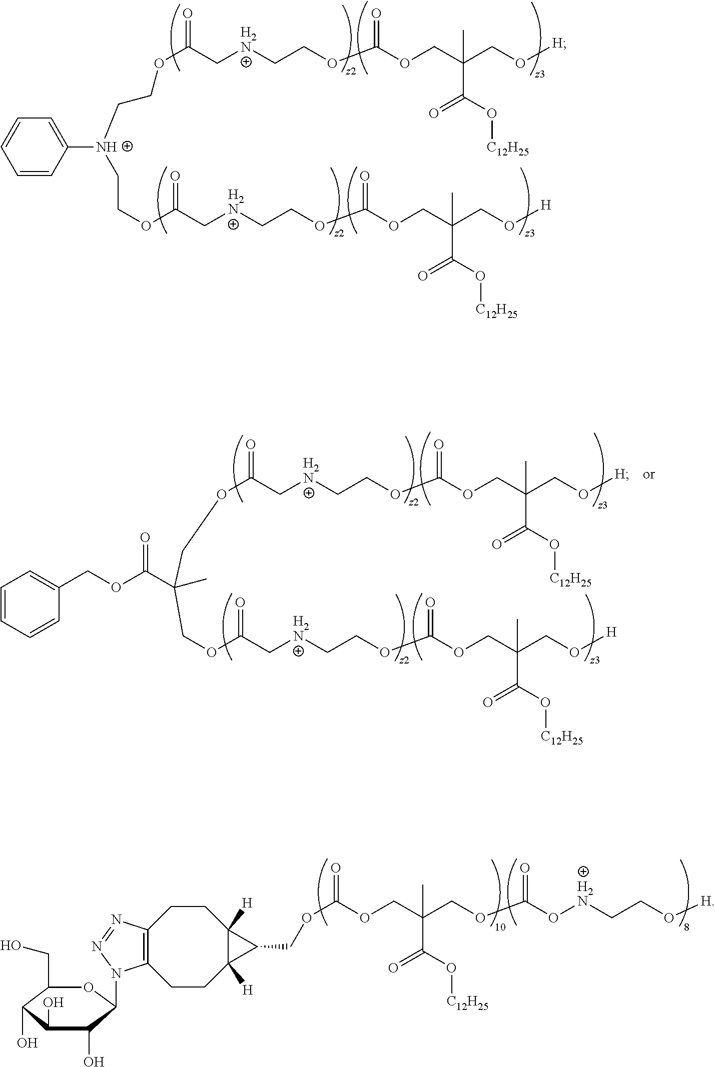

14. A cell penetrating complex comprising a nucleic acid non-covalently attached to the copolymer of claim 1.

15. A pharmaceutical composition comprising the cell-penetrating complex of claim 14.

16. A method of transfecting a nucleic acid into a cell, the method comprising contacting a cell with the cell penetrating complex of claim 14, wherein the cell expresses a receptor on its surface that binds to the ligand moiety of the cell-penetrating complex, wherein the contacting is performed ex vivo or in vivo.

17. The method of claim 16, the method comprising contacting a cell with a cell penetrating complex comprising a nucleic acid non-covalently attached to the copolymer of claim 6.

18. The method of claim 17, where the cell penetrating complex comprises an mRNA non-covalently attached to the copolymer.

19. The method of claim 18, where the cell penetrating complex comprises an mRNA non-covalently attached to the copolymer of claim 11.

20. The method of claim 19, where the cell is a B or T lymphocyte.

21. The method of claim 20, wherein the cell is a marginal zone B cell, a follicular B cell, or a natural killer cell.

22. A method for treating an autoimmune disease or disorder, a cancer, or an infectious disease, the method comprising administering the pharmaceutical composition of claim 15 to a subject in need of therapy for an autoimmune disease or disorder, a cancer, or an infectious disease.

23. The cell penetrating complex of claim 14, wherein the nucleic acid is an mRNA.

24. The method of claim 22, wherein administration is by a parenteral route.
